(19) United States
(12) Patent Application Publication
(10) Pub. No.: US 2025/0313844 A1
(43) Pub. Date: Oct. 9, 2025

(54) INTERFERING RNA FOR INHIBITING PCSK9 GENE EXPRESSION AND USE THEREOF

(71) Applicant: JENKEM TECHNOLOGY CO., LTD. (BEIJING), Beijing (CN)

(72) Inventors: Mai YANG, Beijing (CN); Meina LIN, Beijing (CN); Hongli JIA, Beijing (CN); Yichen LI, Beijing (CN); Xiaomeng CHEN, Beijing (CN); Jun GUO, Beijing (CN); Qingbin WANG, Beijing (CN); Xuan ZHAO, Beijing (CN)

(21) Appl. No.: 19/172,102

(22) Filed: Apr. 7, 2025

(30) Foreign Application Priority Data
Apr. 9, 2024 (CN) 202410420852.X
Feb. 17, 2025 (CN) 202510169275.6

(51) Int. Cl. C12N 15/113

(52) U.S. Cl. CPC C12N 15/1137; C12Y 304/21; C12N 2310/14; C12N 2310/321; C12N 2310/322; C12N 2310/351

(57) ABSTRACT

The present invention discloses an interfering RNA for inhibiting PCSK9 gene and use thereof. The interfering RNA comprises a nucleotide sequence set forth in any one or two or more of SEQ ID NOs: 1-40, 73-96. The interfering RNA of the present invention can better target and silence hepatic PCSK9 mRNA, reduce the protein level of PCSK9, enhance LDL-C metabolism, and reduce serum cholesterol, providing a solid technical foundation for the development of siRNA medicaments for the prevention, treatment, and symptom alleviation of PCSK9 gene-mediated diseases.

### CROSS-REFERENCE TO RELATED APPLICATIONS

This application claims priority to CN 202410420852.X filed Apr. 9, 2024, and CN 202510169275.6 filed Feb. 17, 2025, the entire contents of each of which are hereby incorporated by reference.

### REFERENCE TO SEQUENCE LISTING SUBMITTED ELECTRONICALLY

The content of the electronically submitted sequence listing (Name 8575-80_sequence listing.xml; Size: 251 KB; and Date of Creation: Mar. 26, 2025) is incorporated by reference in its entirety.

### FIELD OF THE INVENTION

The present invention relates to the technical field of molecular biology and biomedicine, and in particular to an interfering RNA for inhibiting PCSK9 gene expression and use thereof.

### BACKGROUND OF THE INVENTION

Low-density lipoprotein cholesterol (LDL-C) is one of the important factors leading to atherosclerotic cardiovascular disease, and LDL-C degradation mediated by low-density lipoprotein receptor (LDL-R) on the surface of hepatocytes is a key pathway for regulating cholesterol levels. The extracellular domain of LDL-R can bind to LDL-C in the bloodstream, and the resulting complex is internalized into the cell via clathrin-coated vesicles. Within the acidic endosomes, LDL-R dissociates from LDL-C, and with the assistance of other proteins, LDL-R is recycled back to the plasma membrane for reuse, while LDL-C is degraded in lysosomes. Binding of proprotein convertase subtilisin/kexin type 9 (PCSK9) to LDL-R can cause the latter to enter lysosomes for degradation, thereby reducing the level of LDL-R on the surface of hepatocytes and affecting the metabolism of LDL-C in the blood. Studies have shown that gain-of-function mutations in PCSK9 promote the degradation of LDL-R in the liver, resulting in a typical familial hypercholesterolemia phenotype, while loss-of-function mutations increase LDL-R activity and are associated with a reduced plasma LDL-C level and a decreased risk of cardiovascular disease (Abifadel et al., 2003; Leigh et al., 2009). The discovery of the interaction between PCSK9 and LDL-R has led to another step forward in our understanding of the pathology of dyslipidemia and laid the foundation for the idea of treating related diseases by limiting the PCSK9-LDL-R interaction.

Current medicaments targeting PCSK9 include Evolocumab from Amgen, Alirocumab from Sanofi and Regeneron, and Leqvio® (Inclisiran) from Novartis. Among them, the first two medicaments are monoclonal antibody medicaments, and Inclisiran is an siRNA medicament. siRNA (small interfering RNA) is a double-stranded RNA with a length of about 19-23 nucleotides. In vivo, the siRNA duplex unwinds and assembles with other proteins to form an RNA-induced silencing complex (RISC); through base complementary pairing, this complex targets a specific mRNA for degradation, thereby achieving the purpose of inhibiting protein expression. This mechanism is called RNA interference (RNAi). Compared to antibody medicaments, siRNA medicaments bypass restrictions of protein spatial structure and theoretically can interfere with the expression of any gene. Therefore, RNAi provides new research and development ideas for many “undruggable” targets.

Although one siRNA medicament targeting PCSK9 is currently on the market, its interference efficacy, safety, and stability vary considerably depending on different siRNA sequences, types of modification, and delivery systems. Therefore, the development of a new medicament with better efficacy, improved stability, and higher safety is of great significance.

### SUMMARY OF THE INVENTION

In order to better target and silence hepatic PCSK9 mRNA, reduce the protein level of PCSK9, enhance LDL-C metabolism, and reduce serum cholesterol, thereby developing a PCSK9-targeting medicament with better efficacy and higher safety, the present invention provides the following technical solutions.

In a first aspect, the present invention provides an interfering RNA for inhibiting PCSK9 gene expression, which comprises a nucleotide sequence set forth in any one or two or more of SEQ ID NOs: 1-40, 73-96.

Specifically, the nucleotide sequences set forth in SEQ ID NOs: 1-40 are as follows:

| Name | Sense strand 5′-3′ | Antisense strand 5′-3′ |
|---|---|---|
| siPCSK9-518 | CCUACGUGGUGGUGCUGAAGG (SEQ | UUCAGCACCACCACGUAGGUG (SEQ |
| | ID NO: 1) | ID NO: 21) |
| siPCSK9-614 | CCAAGAUCCUGCAUGUCUUCC (SEQ | AAGACAUGCAGGAUCUUGGUG (SEQ |
| | ID NO: 2) | ID NO: 22) |
| siPCSK9-1068 | GGUUAGCGGCACCCUCAUAGG (SEQ | UAUGAGGGUGCCGCUAACCGU (SEQ |
| | ID NO: 3) | ID NO: 23) |
| siPCSK9-1163 | GGUACAGCCGCGUCCUCAACG (SEQ | UUGAGGACGCGGCUGUACCCA (SEQ |
| | ID NO: 4) | ID NO: 24) |
| siPCSK9-1415 | GCACCUGCUUUGUGUCACAGA (SEQ | UGUGACACAAAGCAGGUGCUG (SEQ |
| | ID NO: 5) | ID NO: 25) |
| siPCSK9-2102 | GUCUGGAAUGCAAAGUCAAGG (SEQ | UUGACUUUGCAUUCCAGACCU (SEQ |
| | ID NO: 6) | ID NO: 26) |
| siPCSK9-2879 | CCUGAUUAAUGGAGGCUUAGC (SEQ | UAAGCCUCCAUUAAUCAGGGA (SEQ |
| | ID NO: 7) | ID NO: 27) |
| siPCSK9-3059 | GGCAGUGUGCAGUGGUGCAUG (SEQ | UGCACCACUGCACACUGCCGA (SEQ |
| | ID NO: 8) | ID NO: 28) |
| siPCSK9-3131 | CCUACUUCACAGAGGAAGAAA (SEQ | UCUUCCUCUGUGAAGUAGGGG (SEQ |
| | ID NO: 9) | ID NO: 29) |
| siPCSK9-3140 | CAGAGGAAGAAACCUGGAACC (SEQ | UUCCAGGUUUCUUCCUCUGUG (SEQ |
| | ID NO: 10) | ID NO: 30) |
| siPCSK9-3534 | CCUGUUUUGCUUUUGUAACUU (SEQ | AAGUUACAAAAGCAAAACAGGUC |
| | ID NO: 11) | (SEQ ID NO: 31) |
| siPCSK9-3536 | UGUUUUGCUUUUGUAACUUGA (SEQ | UCAAGUUACAAAAGCAAAACAGG |
| | ID NO: 12) | (SEQ ID NO: 32) |
| siPCSK9-3549 | UUUGCUUUUGUAACUUGAAGA (SEQ | UCUUCAAGUUACAAAAGCAAAAC |
| | ID NO: 13) | (SEQ ID NO: 33) |
| siPCSK9-3541 | UGCUUUUGUAACUUGAAGAUA (SEQ | UAUCUUCAAGUUACAAAAGCAAA |
| | ID NO: 14) | (SEQ ID NO: 34) |
| siPCSK9-3542 | GCUUUUGUAACUUGAAGAUAU (SEQ | AUAUCUUCAAGUUACAAAAGCAA |
| | ID NO: 15) | (SEQ ID NO: 35) |
| siPCSK9-3549 | UAACUUGAAGAUAUUUAUUCU (SEQ | AGAAUAAAUAUCUUCAAGUUACA |
| | ID NO: 16) | (SEQ ID NO: 36) |
| siPCSK9-3553 | UUGAAGAUAUUUAUUCUGGGU (SEQ | ACCCAGAAUAAAUAUCUUCAAGU |
| | ID NO: 17) | (SEQ ID NO: 37) |
| siPCSK9-3554 | UGAAGAUAUUUAUUCUGGGUU (SEQ | AACCCAGAAUAAAUAUCUUCAAG |
| | ID NO: 18) | (SEQ ID NO: 38) |
| siPCSK9-3556 | AAGAUAUUUAUUCUGGGUUUU (SEQ | AAAACCCAGAAUAAAUAUCUUCA |
| | ID NO: 19) | (SEQ ID NO: 39) |
| siPCSK9-3558 | GAUAUUUAUUCUGGGUUUUGU (SEQ | ACAAAACCCAGAAUAAAUAUCUU |
| | ID NO: 20) | (SEQ ID NO: 40) |

Specifically, the nucleotide sequences set forth in SEQ ID NOs: 73-96 are as follows:

| Name | Sense strand 5′-3′ | Antisense strand 5′-3′ |
|---|---|---|
| siPCSK9- | CmsCmsAmAmGmAm(Uf)Cm(Cf)Um(Gf)CmA | Ams(Af)s(Gf)(Af)Cm(Af)Um(Gf)Cm(Af)Gm |
| 614-m1-L96 | mUmGmUmCmUmUmCmCmL96 (SEQ ID | (Gf)Am(Uf)Cm(Uf)UmGmGmsUmsGm (SEQ ID |
| | NO: 73) | NO: 85) |
| siPCSK9- | CmsCmsAmAmGmAm(Uf)Cm(Cf)(Uf)(Gf)CmA | AmsAmsGm(Af)Cm(Af)(Uf)GmCmAmGm(Gf) |
| 614-m2-L96 | mUmGmUmCmUmUmCmCmL96 (SEQ ID | Am(Uf)CmUmUmGmGmsUmsGm (SEQ ID |
| | NO: 74) | NO: 86) |
| siPCSK9- | GmsUmsCmUmGmGm(Af)Am(Uf)Gm(Cf)AmA | Ums(Uf)s(Gf)(Af)Cm(Uf)Um(Uf)Gm(Cf)Am |
| 2102-m1- | mAmGmUmCmAmAmGmGmL96 (SEQ ID | (Uf)Um(Cf)Cm(Af)GmAmCmsCmsUm (SEQ ID |
| L96 | NO: 75) | NO: 87) |
| siPCSK9- | GmsUmsCmUmGmGm(Af)Am(Uf)(Gf)(Cf)Am | UmsUmsGm(Af)Cm(Uf)(Uf)UmGmCmAm(Uf) |
| 2102-m2- | AmAmGmUmCmAmAmGmGmL96 (SEQ ID | Um(Cf)CmAmGmAmCmsCmsUm (SEQ ID |
| L96 | NO: 76) | NO: 88) |
| siPCSK9- | CmsCmsUmGmUmUm(Uf)Um(Gf)Cm(Uf)UmU | Ams(Af)sGm(Uf)(Uf)(Af)Cm(Af)Am(Af)Am |
| 3534-m1- | mUmGmUmAmAmCmUmUmL96 (SEQ ID | (Gf)Cm(Af)Am(Af)Am(Cf)AmGmGmsUmsCm |
| L96 | NO: 77) | (SEQ ID NO: 89) |
| siPCSK9- | CmsCmsUmGmUmUm(Uf)Um(Gf)(Cf)(Uf)Um | Ams(Af)sGmUmUm(Af)Cm(Af)(Af)AmAmGm |
| 3534-m2- | UmUmGmUmAmAmCmUmUmL96 (SEQ ID | Cm(Af)Am(Af)AmCmAmGmGmsUmsCm (SEQ |
| L96 | NO: 78) | ID NO: 90) |
| siPCSK9- | UmsGmsUmUmUmUm(Gf)Cm(Uf)Um(Uf)UmG | Ums(Cf)sAm(Af)(Gf)(Uf)Um(Af)Cm(Af)Am |
| 3536-m1- | mUmAmAmCmUmUmGmAmL96 (SEQ ID | (Af)Am(Gf)Cm(Af)Am(Af)AmCmAmsGmsGm |
| L96 | NO: 79) | (SEQ ID NO: 91) |
| siPCSK9- | UmsGmsUmUmUmUm(Gf)Cm(Uf)(Uf)(Uf)Um | Ums(Cf)sAmAmGm(Uf)Um(Af)(Cf)AmAmAm |
| 3536-m2- | GmUmAmAmCmUmUmGmAmL96 (SEQ ID | Am(Gf)Cm(Af)AmAmAmCmAmsGmsGm (SEQ |
| L96 | NO: 80) | ID NO: 92) |
| siPCSK9- | UmsGmsAmAmGmAm(Uf)Am(Uf)Um(Uf)Am | Ams(Af)sCm(Cf)(Cf)(Af)Gm(Af)Am(Uf)Am |
| 3554-m1- | UmUmCmUmGmGmGmUmUmL96 (SEQ ID | (Af)Am(Uf)Am(Uf)Cm(Uf)UmCmAmsAmsGm |
| L96 | NO: 81) | (SEQ ID NO: 93) |
| siPCSK9- | UmsGmsAmAmGmAm(Uf)Am(Uf)(Uf)(Uf)Am | Ams(Af)sCmCmCm(Af)Gm(Af)(Af)UmAmAm |
| 3554-m2- | UmUmCmUmGmGmGmUmUmL96 (SEQ ID | Am(Uf)Am(Uf)CmUmUmCmAmsAmsGm (SEQ |
| L96 | NO: 82) | ID NO: 94) |
| siPCSK9- | AmsAmsGmAmUmAm(Uf)Um(Uf)Am(Uf)UmC | Ams(Af)sAm(Af)(Cf)(Cf)Cm(Af)Gm(Af)Am |
| 3556-m1- | mUmGmGmGmUmUmUmUmL96 (SEQ ID | (Uf)Am(Af)Am(Uf)Am(Uf)CmUmUmsCmsAm |
| L96 | NO: 83) | (SEQ ID NO: 95) |
| siPCSK9- | AmsAmsGmAmUmAm(Uf)Um(Uf)(Af)(Uf)Um | Ams(Af)sAmAmCm(Cf)Cm(Af)(Gf)AmAmUm |
| 3556-m2- | CmUmGmGmGmUmUmUmUmL96 (SEQ ID | Am(Af)Am(Uf)AmUmCmUmUmsCmsAm (SEQ |
| L96 | NO: 84) | ID NO: 96) |

Furthermore, the interfering RNA comprises at least one of the following combinations of sense and antisense strands:

a combination of SEQ ID NO: 1 and SEQ ID NO: 21, a combination of SEQ ID NO: 2 and SEQ ID NO: 22, a combination of SEQ ID NO: 3 and SEQ ID NO: 23, a combination of SEQ ID NO: 4 and SEQ ID NO: 24, a combination of SEQ ID NO: 5 and SEQ ID NO: 25, a combination of SEQ ID NO: 6 and SEQ ID NO: 26, a combination of SEQ ID NO: 7 and SEQ ID NO: 27, a combination of SEQ ID NO: 8 and SEQ ID NO: 28, a combination of SEQ ID NO: 9 and SEQ ID NO: 29, a combination of SEQ ID NO: 10 and SEQ ID NO: 30, a combination of SEQ ID NO: 11 and SEQ ID NO: 31, a combination of SEQ ID NO: 12 and SEQ ID NO: 32, a combination of SEQ ID NO: 13 and SEQ ID NO: 33, a combination of SEQ ID NO: 14 and SEQ ID NO: 34, a combination of SEQ ID NO: 15 and SEQ ID NO: 35, a combination of SEQ ID NO: 16 and SEQ ID NO: 36, a combination of SEQ ID NO: 17 and SEQ ID NO: 37, a combination of SEQ ID NO: 18 and SEQ ID NO: 38, a combination of SEQ ID NO: 19 and SEQ ID NO: 39, a combination of SEQ ID NO: 20 and SEQ ID NO: 40, a combination of SEQ ID NO:73 and SEQ ID NO:85, a combination of SEQ ID NO:74 and SEQ ID NO:86, a combination of SEQ ID NO:75 and SEQ ID NO:87, a combination of SEQ ID NO: 76 and SEQ ID NO:88, a combination of SEQ ID NO:77 and SEQ ID NO:89, a combination of SEQ ID NO:78 and SEQ ID NO:90, a combination of SEQ ID NO:79 and SEQ ID NO:91, a combination of SEQ ID NO:80 and SEQ ID NO:92, a combination of SEQ ID NO:81 and SEQ ID NO: 93, a combination of SEQ ID NO:82 and SEQ ID NO:94, a combination of SEQ ID NO:83 and SEQ ID NO:95, and a combination of SEQ ID NO:84 and SEQ ID NO:96.

Preferably, the interfering RNA molecule may further comprise at least one modified nucleotide, wherein the modification includes a modification on a base, a modification on a sugar ring, and/or a modification on a phosphate backbone.

Preferably, the modification on a base includes, but is not limited to, pyrimidine modification at position 5, purine modification at position 8, pseudouridine modification, and/or 5-bromouracil substitution.

Preferably, the modification on a sugar ring includes, but is not limited to, 2′-hydroxyl modification, 2′-fluoro modification, 2′-deoxy modification, 2′-O-methyl modification, 2′-O-methoxyethyl modification, 2′-O-allyl modification, 2′-C-allyl modification, and locked nucleic acid modification.

Preferably, the modification on a phosphate backbone includes, but is not limited to, phosphorothioate modification and ligand modification.

Preferably, the ligand modification includes, but is not limited to, cholesterol, biotin, a vitamin, a galactose derivative or analog, a lactose derivative or analog, N-acetylgalactosamine (GalNAc), an N-acetylgalactosamine derivative or analog, an N-acetylglucosamine derivative or analog, a mannose 6-phosphate (M6P) derivative or analog, and any combination thereof.

Preferably, the GalNAc derivative or analog has a structural formula as follows:

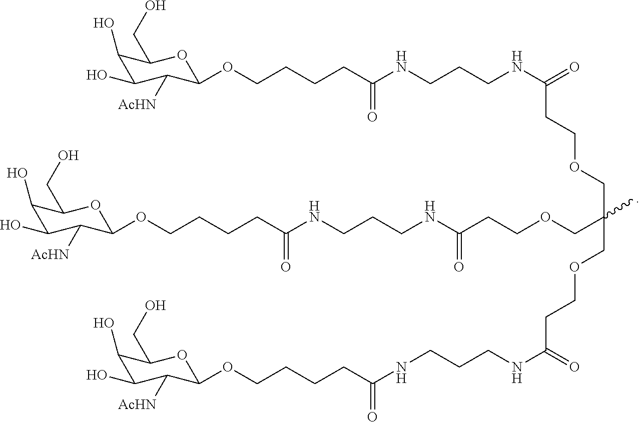 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

The interfering RNA has a structural formula as follows:

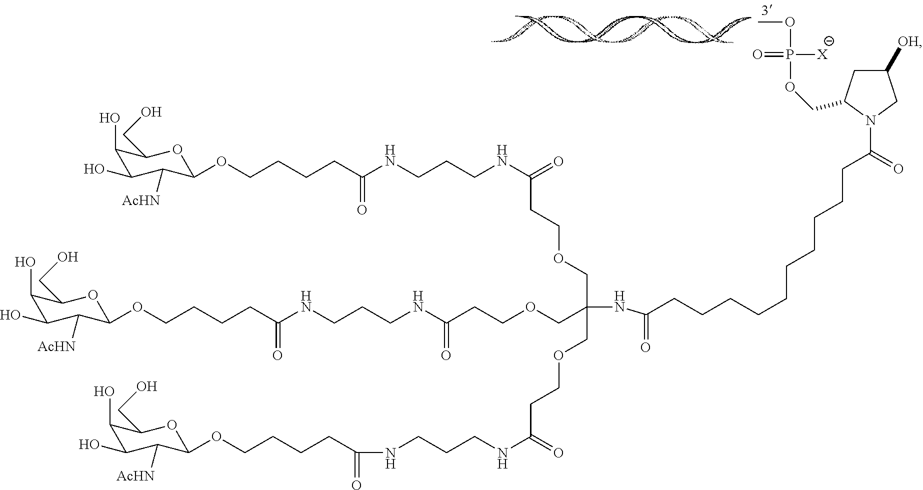

wherein X represents S or O.

In a second aspect, the present invention provides a delivery system for an interfering RNA, which comprises the interfering RNA according to the first aspect and a vector.

Preferably, the vector is a viral vector or a non-viral vector;

more preferably, the viral vector includes one or a combination of two or more of a lentivirus vector, a retrovirus vector, an adenovirus vector, an adeno-associated virus vector, a poxvirus vector, or a herpesvirus vector;

more preferably, the non-viral vector includes any one or a combination of two or more of a liposome, a lipid nanoparticle, a polymer, a polypeptide, an antibody, or an aptamer.

Furthermore, the lipid nanoparticle or the liposome includes one or a combination of two or more of a cationic lipid, a neutral lipid, a polyethylene glycol lipid, a steroidal lipid, or an anionic lipid.

Furthermore, the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl) azanediyl] di(hexane-6,1-diyl)bis(2-hexyldecanoate) (ALC-0315), 1,2-dioleoyloxy-3-(trimethylammonium) propane (DOTAP), 1,2-di-(9Z-octadecenoyl)-3-trimethylammonium-propane and 1,2-dihexadecanoyl-3-trimethylammonium-propane, 3B-[N-(N′,N′-dimethylaminoethane)-carbamoyl] cholesterol (DC-cholesterol), dimethyldioctadecylammonium (DDA), 1,2-dimyristoyl-3-trimethylammonium propane (DMTAP), dipalmitoyl(C16: 0)trimethylammonium propane (DPTAP), distearoyltrimethylammonium propane (DSTAP), N-[1-(2,3-diallyloxy) propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2 (spermine-carboxamido)ethyl]-N,N-dimethyl-1-trifluoroacetate propanaminium (DOSPA)

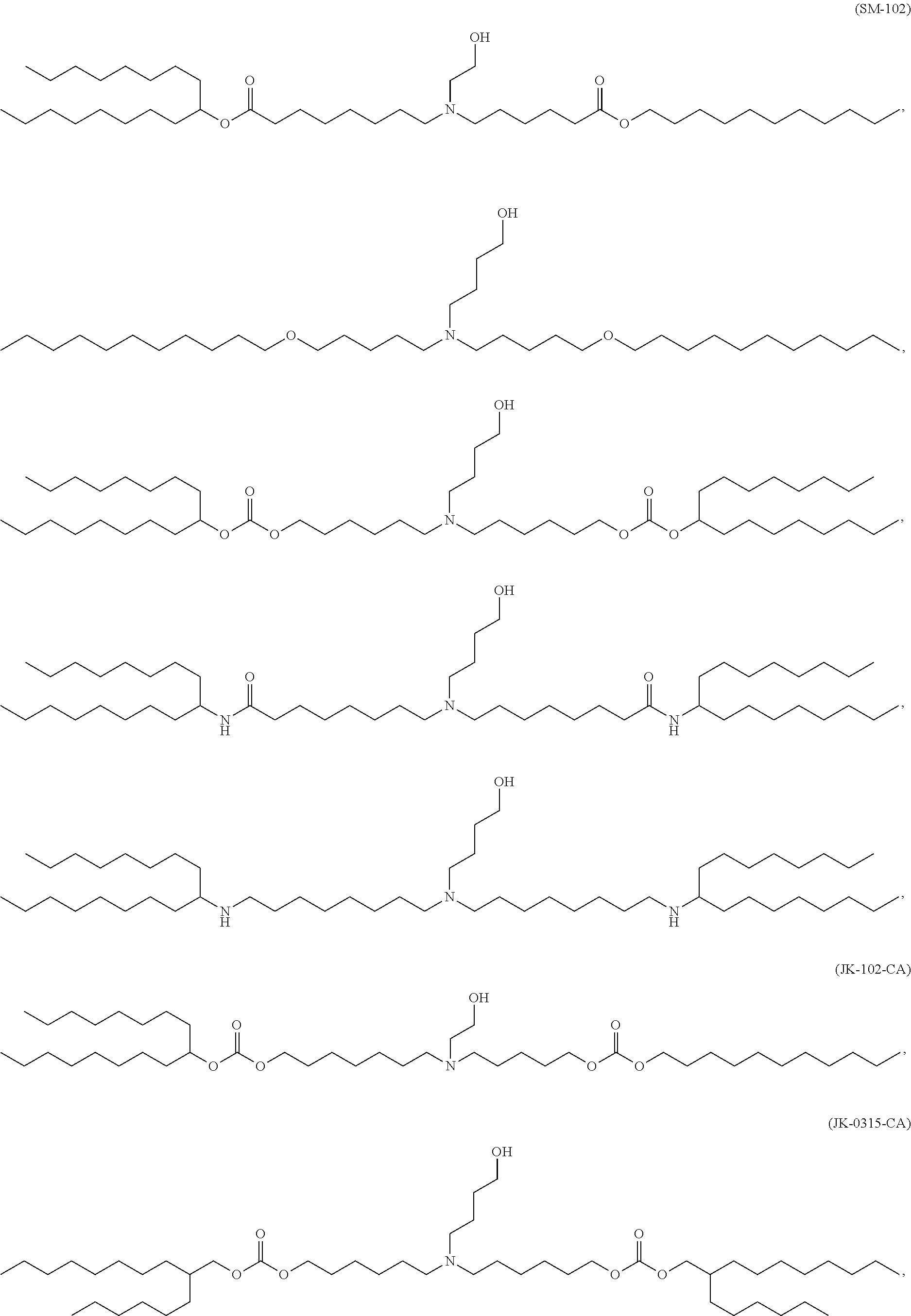

### and Dlin-MC3-DMA.

Furthermore, the neutral lipid includes one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

Furthermore, the polyethylene glycol lipid includes one or a combination of two or more of 2-[(polyethylene glycol)-2000]-N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA),

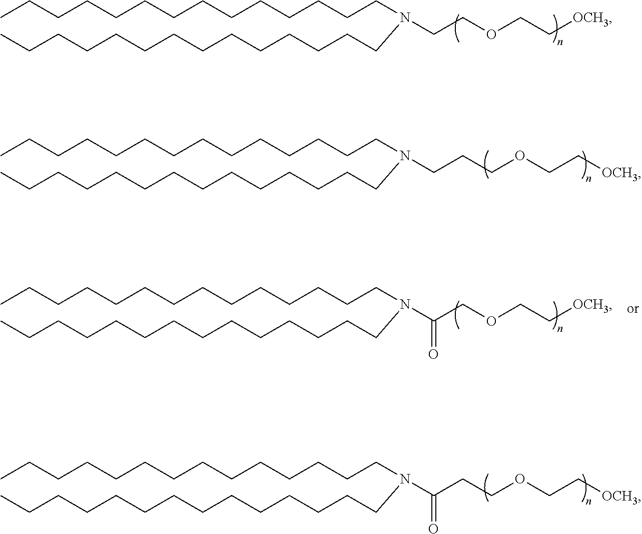

wherein n is selected from integers of 20-300.

Furthermore, the polyethylene glycol lipid is a polyethylene glycol lipid with a single molecular weight, and preferably the polyethylene glycol lipid includes:

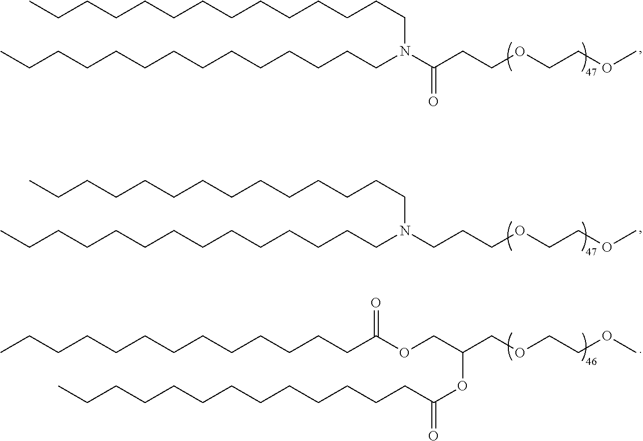

Furthermore, the cationic lipid is a steroid-cationic lipid compound, and the compound has a structure of:

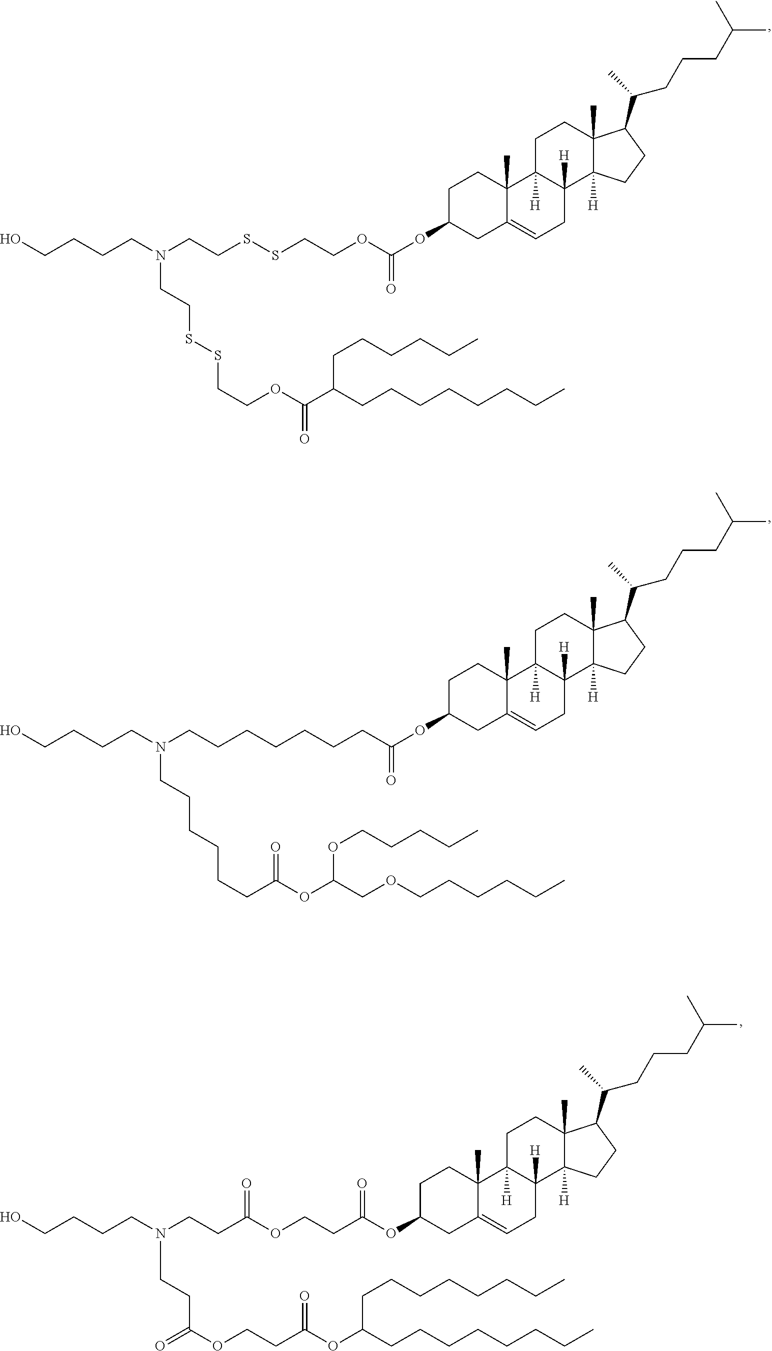

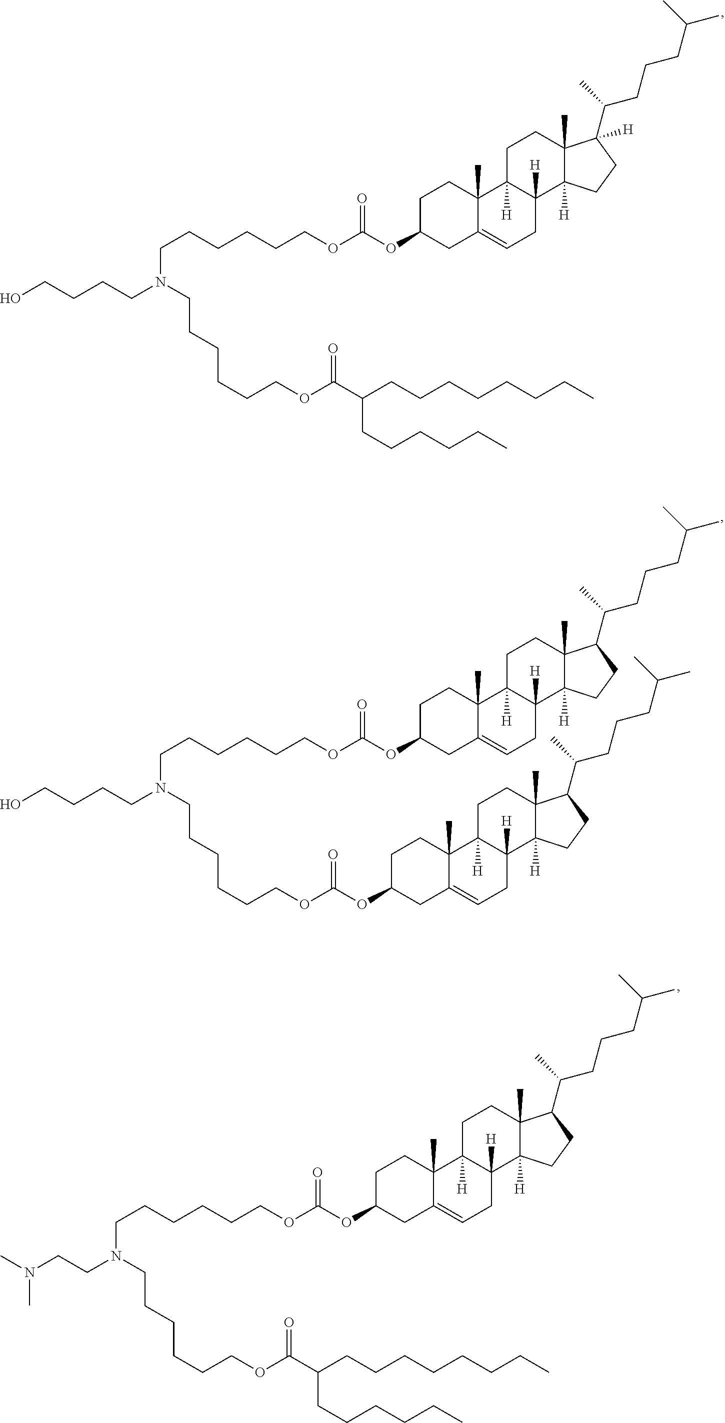

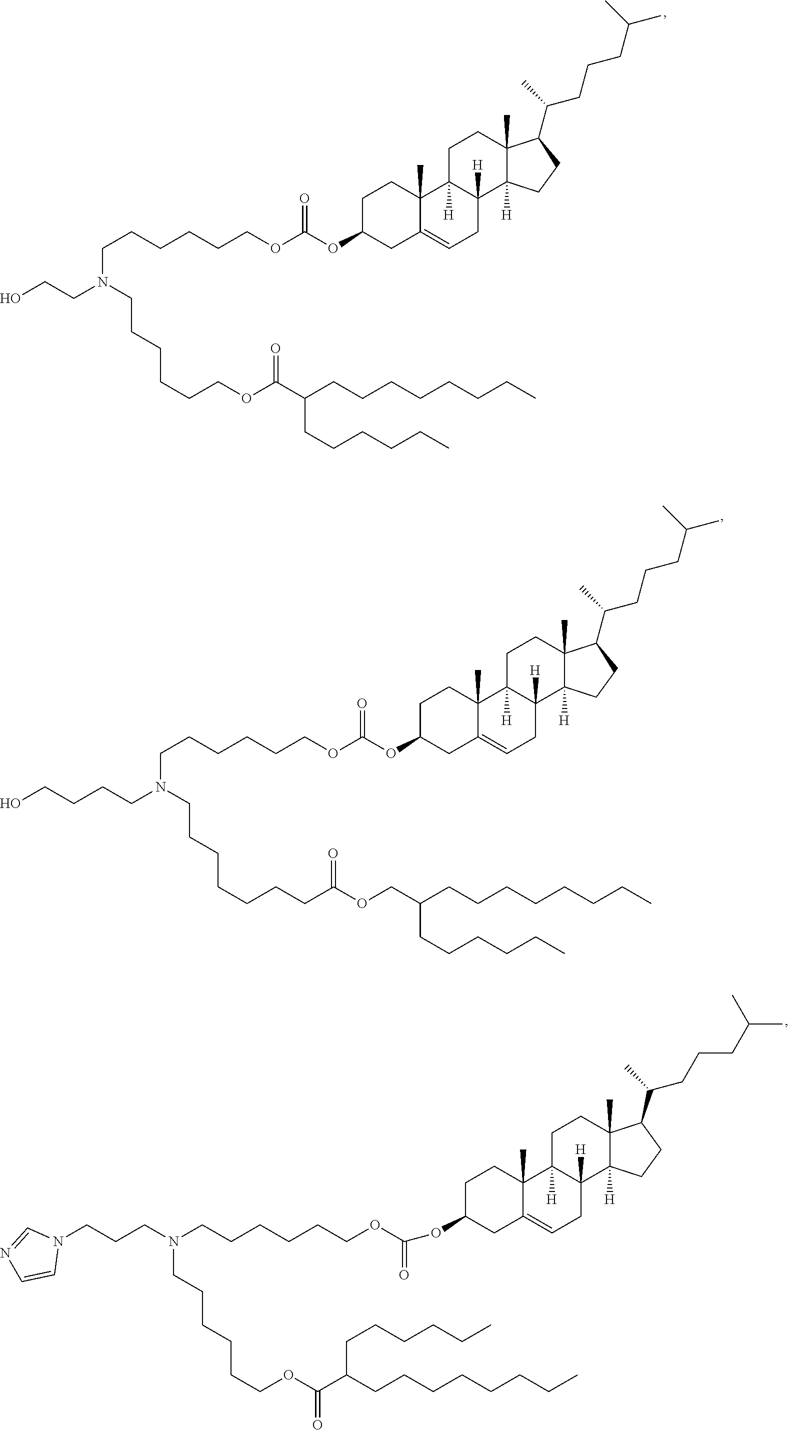

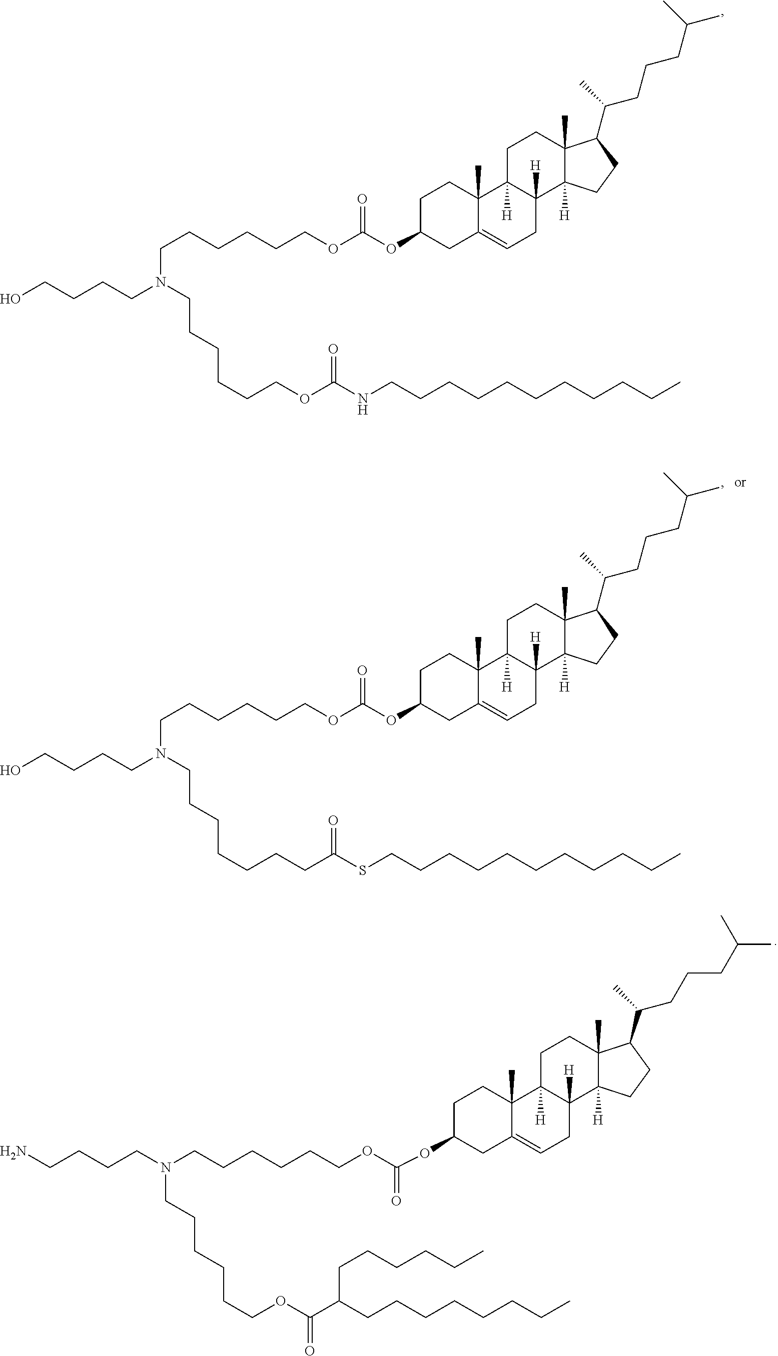

Furthermore, the anionic liposome includes one or a combination of two or more of dioleoyl phosphatidylglycerol or dioleoyl phosphatidylethanolamine.

Furthermore, the steroidal lipid includes one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid.

In a third aspect, the present invention provides a medicament or kit, which comprises the interfering RNA according to the first aspect or the delivery system according to the second aspect.

In a fourth aspect, the present invention provides use of the interfering RNA according to the first aspect or the delivery system according to the second aspect.

Preferably, the use includes the following:
(1) use in inhibiting PCSK9 gene expression or in preparing a product for inhibiting PCSK9 gene expression;
(2) use in reducing the concentration of low-density lipoprotein and/or low-density lipoprotein cholesterol in a serum or in preparing a product for reducing the concentration of low-density lipoprotein and/or low-density lipoprotein cholesterol in a serum;
(3) use in preventing and/or treating a PCSK9 gene-mediated disease or in preparing a product for preventing and/or treating a PCSK9 gene-mediated disease; and
(4) use in alleviating a symptom of a PCSK9 gene-mediated disease or in preparing a product for alleviating a symptom of a PCSK9 gene-mediated disease.

Preferably, the PCSK9 gene-mediated disease includes a cardiovascular disease or a neoplastic disease.

Furthermore, the cardiovascular disease includes hyperlipidemia, hypercholesterolemia, non-familial hypercholesterolemia, polygenic hypercholesterolemia, familial hypercholesterolemia, homozygous familial hypercholesterolemia, heterozygous familial hypercholesterolemia, and mixed dyslipidemia in mammals;

Furthermore, the neoplastic disease includes PCSK9-associated melanoma or metastatic liver cancer.

The present invention has the following beneficial effects:

The interfering RNA of the present invention can stably and efficiently inhibit PCSK9 expression, thereby providing a solid technical foundation for the development of siRNA medicaments for the prevention, treatment, and symptom alleviation of PCSK9 gene-mediated diseases, and enriching treatment means for related diseases.

### BRIEF DESCRIPTION OF THE DRAWINGS

FIG. 1 shows the inhibitory effect of siRNA on protein in transgenic mice.

### DETAILED DESCRIPTION OF THE INVENTION

The technical solutions of the present invention will be clearly and completely described with reference to the following examples. The described examples are only a part of the examples of the present invention, but not all of them. Based on the examples of the present invention, other examples obtained by those of ordinary skill in the art without creative work shall fall within the protection scope of the present invention.

Unless otherwise defined, all scientific and technical terms used in the present invention have the same meaning as commonly understood by those skilled in the art to which the present invention relates.

### Example 1. siRNA Design and Synthesis

siRNAs were designed according to the mRNA sequence of human PCSK9, which was derived from the NCBI database (https://www.ncbi.nlm.nih.gov/gene/). The target sequences and siRNA sequences are shown in Table 1. The siRNA sequences were synthesized by Beijing Tsingke Biotech Co., Ltd.

The unrelated sequences (siNC, sense strand: 5′-UUCUCCGAACGUGUCACGUTT-3′ (SEQ ID NO: 61), antisense strand: 5′-ACGUGACACGUUCGGAGAATT-3′ (SEQ ID NO: 62)) were designed and synthesized by Sangon Biotech (Shanghai) Co., Ltd.

Inclisiran (sense strand: 5′-CmsUmsAmGmAmCmCfUmGfUmdTUmUmGmCmUmUmUmUmGmUmL96-3′ (SEQ ID NO: 63), antisense strand: 5′-AmsCfsAmAfAfAfGmCfAmAfAmAfCmAfGmGfUmCfUmAmGmsAmsAm-3′ (SEQ ID NO: 64), wherein m represents 2′-OMe modification on the adjacent nucleotide to the left, f represents 2′-F modification on the adjacent nucleotide to the left, s represents phosphorothioate modification on the phosphate backbone, dT represents thymine, and L96 represents N-acetylgalactosamine (GalNAc), which is Leqvio® from Novartis, was synthesized by Sangon Biotech (Shanghai) Co., Ltd.

siPCSK9-3267 (sense strand: 5′-CAUUUUUACGGGUAACAGUGA-3′ (SEQ ID NO: 65), antisense strand: 5′-ACUGUUACCCGUAAAAAUGAG-3′ (SEQ ID NO: 66)) and siPCSK9-3580 (sense strand: 5′-GCAUUUUUAUUAAUAUGGUGA-3′ (SEQ ID NO: 67), antisense strand: 5′-ACCAUAUUAAUAAAAAUGCUA-3′ (SEQ ID NO: 68)), which are existing known siRNAs (CN114716518A), were synthesized by Beijing Tsingke Biotech Co., Ltd.

TABLE 1
Target sequences and siRNA sequences

| Name | Target sequence | Sense strand 5′-3′ | Antisense strand 5′-3′ |
|---|---|---|---|
| siPCSK9- | CACCTACGTGGTGGTGCTGA | CCUACGUGGUGGUGCUGA | UUCAGCACCACCACGUAGG |
| 518 | AGG (SEQ ID NO: 41) | AGG (SEQ ID NO: 1) | UG (SEQ ID NO: 21) |
| siPCSK9- | CACCAAGATCCTGCATGTCTT | CCAAGAUCCUGCAUGUCU | AAGACAUGCAGGAUCUUG |
| 614 | CC (SEQ ID NO: 42) | UCC (SEQ ID NO: 2) | GUG (SEQ ID NO: 22) |
| siPCSK9- | ACGGTTAGCGGCACCCTCAT | GGUUAGCGGCACCCUCAU | UAUGAGGGUGCCGCUAAC |
| 1068 | AGG (SEQ ID NO: 43) | AGG (SEQ ID NO: 3) | CGU (SEQ ID NO: 23) |
| siPCSK9- | TGGGTACAGCCGCGTCCTCA | GGUACAGCCGCGUCCUCA | UUGAGGACGCGGCUGUAC |
| 1163 | ACG (SEQ ID NO: 44) | ACG (SEQ ID NO: 4) | CCA (SEQ ID NO: 24) |
| siPCSK9- | CAGCACCTGCTTTGTGTCAC | GCACCUGCUUUGUGUCAC | UGUGACACAAAGCAGGUG |
| 1415 | AGA (SEQ ID NO: 45) | AGA (SEQ ID NO: 5) | CUG (SEQ ID NO: 25) |
| siPCSK9- | AGGTCTGGAATGCAAAGTCA | GUCUGGAAUGCAAAGUCA | UUGACUUUGCAUUCCAGA |
| 2102 | AGG (SEQ ID NO: 46) | AGG (SEQ ID NO: 6) | CCU (SEQ ID NO: 26) |
| siPCSK9- | TCCCTGATTAATGGAGGCTTA | CCUGAUUAAUGGAGGCUU | UAAGCCUCCAUUAAUCAG |
| 2879 | GC (SEQ ID NO: 47) | AGC (SEQ ID NO: 7) | GGA (SEQ ID NO: 27) |
| siPCSK9- | TCGGCAGTGTGCAGTGGTGC | GGCAGUGUGCAGUGGUGC | UGCACCACUGCACACUGCC |
| 3059 | ATG (SEQ ID NO: 48) | AUG (SEQ ID NO: 8) | GA (SEQ ID NO: 28) |
| siPCSK9- | CCCCTACTTCACAGAGGAAG | CCUACUUCACAGAGGAAG | UCUUCCUCUGUGAAGUAG |
| 3131 | AAA (SEQ ID NO: 49) | AAA (SEQ ID NO: 9) | GGG (SEQ ID NO: 29) |
| siPCSK9- | CACAGAGGAAGAAACCTGG | CAGAGGAAGAAACCUGGA | UUCCAGGUUUCUUCCUCU |
| 3140 | AACC (SEQ ID NO: 50) | ACC (SEQ ID NO: 10) | GUG (SEQ ID NO: 30) |
| siPCSK9- | GACCTGTTTTGCTTTTGTAAC | CCUGUUUUGCUUUUGUAA | AAGUUACAAAAGCAAAAC |
| 3534 | TT (SEQ ID NO: 51) | CUU (SEQ ID NO: 11) | AGGUC (SEQ ID NO: 31) |
| siPCSK9- | CCTGTTTTGCTTTTGTAACTT | UGUUUUGCUUUUGUAACU | UCAAGUUACAAAAGCAAA |
| 3536 | GA (SEQ ID NO: 52) | UGA (SEQ ID NO: 12) | ACAGG (SEQ ID NO: 32) |
| siPCSK9- | GTTTTGCTTTTGTAACTTGAA | UUUGCUUUUGUAACUUGA | UCUUCAAGUUACAAAAGC |
| 3539 | GA (SEQ ID NO: 53) | AGA (SEQ ID NO: 13) | AAAAC (SEQ ID NO: 33) |
| siPCSK9- | TTTGCTTTTGTAACTTGAAGA | UGCUUUUGUAACUUGAAG | UAUCUUCAAGUUACAAAA |
| 3541 | TA (SEQ ID NO: 54) | AUA (SEQ ID NO: 14) | GCAAA (SEQ ID NO: 34) |
| siPCSK9- | TTGCTTTTGTAACTTGAAGAT | GCUUUUGUAACUUGAAGA | AUAUCUUCAAGUUACAAA |
| 3542 | AT (SEQ ID NO: 55) | UAU (SEQ ID NO: 15) | AGCAA (SEQ ID NO: 35) |
| siPCSK9- | TGTAACTTGAAGATATTTATT | UAACUUGAAGAUAUUUAU | AGAAUAAAUAUCUUCAAG |
| 3549 | CT (SEQ ID NO: 56) | UCU (SEQ ID NO: 16) | UUACA (SEQ ID NO: 36) |
| siPCSK9- | ACTTGAAGATATTTATTCTGG | UUGAAGAUAUUUAUUCUG | ACCCAGAAUAAAUAUCUU |
| 3553 | GT (SEQ ID NO: 57) | GGU (SEQ ID NO: 17) | CAAGU (SEQ ID NO: 37) |
| siPCSK9- | CTTGAAGATATTTATTCTGGG | UGAAGAUAUUUAUUCUGG | AACCCAGAAUAAAUAUCU |
| 3554 | TT (SEQ ID NO: 58) | GUU (SEQ ID NO: 18) | UCAAG (SEQ ID NO: 38) |
| siPCSK9- | TGAAGATATTTATTCTGGGTT | AAGAUAUUUAUUCUGGGU | AAAACCCAGAAUAAAUAU |
| 3556 | TT (SEQ ID NO: 59) | UUU (SEQ ID NO: 19) | CUUCA (SEQ ID NO: 39) |
| siPCSK9- | AAGATATTTATTCTGGGTTTT | GAUAUUUAUUCUGGGUUU | ACAAAACCCAGAAUAAAU |
| 3558 | GT (SEQ ID NO: 60) | UGU (SEQ ID NO: 20) | AUCUU (SEQ ID NO: 40) |

### Example 2: siRNA Screening by In Vitro Cell Experiment

### 2.1. Cell Transfection

One day prior to transfection, Hep3B cells (from Cell Resource Center, IBMS, CAMS/PUMC) were added to a culture plate at 1×10⁵ cells/well, with the culture medium being 500 μL of MEM containing 15% FBS and 1% penicillin-streptomycin, and the culture plate was placed in an incubator at 37° C. with 5% CO₂ and incubated for 24 h. On the day of transfection, the culture medium was changed to one without antibiotics. siRNAs were diluted with 25 μL of Opti-MEM culture medium, and the transfection reagent LipofectamineTMRNAiMAX (Lip) was diluted with another 25 μL of Opti-MEM culture medium. The two dilutions were uniformly mixed, and the mixture was left to stand for 5-10 min at room temperature. The mixture was added to a 24-well cell culture plate at 50 μL/well, with 3 replicate wells set for each group. The resulting mixture was mixed thoroughly. The siRNA working concentration was 10 nM, with the control group receiving siNC at the same concentration. The culture plate was placed in an incubator at 37° C. with 5% CO₂ and incubated for 48 h, and subsequently, the cells were collected for total RNA extraction.

### 2.2. RNA Extraction

RNA was extracted using Eastep™ Super total RNA extraction kit, with the detailed procedures as follows:
1) 300 μL of RNA lysis buffer and 300 μL of RNA diluent were added to each well of the 24-well cell culture plate, and the mixture was uniformly mixed and transferred to a 1.5 mL centrifuge tube. The tube was centrifuged for 10 min at 14000 g, and the supernatant was carefully pipetted into a new 1.5 mL centrifuge tube.
2) A 0.5-fold volume of absolute ethanol was added, and the mixture was uniformly mixed by allowing the tube to be repeatedly upside down 15-20 times. Subsequently, the mixture was transferred to a centrifuge column and centrifuged for 1 min at 12000 g. The filtrate was discarded.
3) 600 μL of RNA wash buffer was added, and the mixture was centrifuged for 1 min at 12000 g. The filtrate was discarded.
4) 50 μL of DNase I incubation buffer was added, followed by DNA digestion for 15 min at room temperature.
5) 600 μL of RNA wash buffer was added again, the mixture was centrifuged for 1 min at 14000 g, and the filtrate was discarded. This procedure was repeated once. The residual RNA wash buffer was removed by centrifugation for 2 min at 14000 g, and the centrifuge column was placed in a collection tube.
6) A proper amount of RNase-free H₂O was added, and the mixture was left to stand for 15 min at room temperature, followed by centrifugation for 1 min at 14000 g. The RNA quality and concentration were detected using NanoDrop™

### 2.3. Detection of Gene Expression Level

1) The extracted RNA was incubated for 5 min at 70° C. and immediately placed in an ice bath for later use.
2) The RNA was reverse-transcribed into a cDNA template using the All-in-One First-Strand Synthesis MasterMix (LABLEAD, Cat. No.: F0202) kit. The reaction system was established according to Table 2, where all components were mixed uniformly and centrifuged to collect the liquid at the bottom of a tube, and the PCR program was run according to the settings of 37° C. for 2 min, 55° C. for 15 min, and 85° C. for 5 min, thereby giving the cDNA template.

TABLE 2
Reverse transcription reaction system

| Reagent | Amount of use | |
|---|---|---|
| RNA | 1 | μg |
| All-in-One First-Strand Synthesis MasterMix | 4 | μL |
| dsDNase | 1 | μL |

| | |
|---|---|
| RNase-free H₂O | Make up to 20 μL |

3) The gene expression level was detected using the Taq SYBR® Green qPCR Premix (LABLEAD, Cat. No.: R0202) kit, and the qPCR system was established according to Table 3:

TABLE 3
qPCR reaction system

| Reagent | Amount of addition | |
|---|---|---|
| cDNA template | 100 | ng |
| Taq SYBR ® Green qPCR Premix | 10 | μL |
| Forward primer (10 μM) | 0.4 | μL |
| Reverse primer (10 μM) | 0.4 | μL |

| | |
|---|---|
| RNase-free H₂O | Make up to 20 μL |

All components were mixed uniformly and centrifuged to collect the liquid at the bottom of a tube, and the PCR program was run according to the settings below:
Step 1:95° C. for 30 s;
Step 2:95° C. for 10 s;
Step 3:60° C. for 30 s+Plate Read;
Step 4: Go to Step 2, 40 more times;
Step 5: Melt Curve 65 to 95° C., increment 0.5° C., for 5 s+Plate Read. GAPDH was used as an endogenous reference to detect the relative expression level of PCSK9 gene, and the sequences of the primers used are as follows:

GAPDH-F:
(SEQ ID NO: 69)
TCTGACTTCAACAGCGACAC;
GAPDH-R:
(SEQ ID NO: 70)
GCCAAATTCGTTGTCATACC;
PCSK9-F:
(SEQ ID NO: 71)
CACAGAGTGGGACATCACAG;
PCSK9-R:
(SEQ ID NO: 72)
TTTGGCAGAGAAGTGGATCAG.

The detection results (see Table 4) demonstrate that when compared to siNC, all the siRNAs targeting PCSK9 designed in Example 1 could significantly inhibit PCSK9 gene expression in Hep3B cells, where siPCSK9-3267 and siPCSK9-3580 exhibited a weaker inhibitory effect on PCSK9 gene expression as compared to other siRNAs.

TABLE 4
Detection of the inhibitory effect of
siPCSK9 on the gene in Hep3B cells

| Hep3B, siRNA | Relative expression level of PCSK9 | |
|---|---|---|
| concentration 10 nM | Mean value (Mean) | Standard deviation (SD) |
| siPCSK9-518 | 0.10 | 0.01 |
| siPCSK9-614 | 0.14 | 0.04 |
| siPCSK9-1068 | 0.13 | 0.03 |
| siPCSK9-1163 | 0.04 | 0.01 |
| siPCSK9-1415 | 0.06 | 0.04 |
| siPCSK9-2102 | 0.12 | 0.04 |
| siPCSK9-2879 | 0.12 | 0.03 |
| siPCSK9-3059 | 0.12 | 0.04 |
| siPCSK9-3131 | 0.18 | 0.07 |
| siPCSK9-3140 | 0.12 | 0.06 |
| siPCSK9-3534 | 0.08 | 0.02 |
| siPCSK9-3536 | 0.10 | 0.04 |
| siPCSK9-3539 | 0.12 | 0.01 |
| siPCSK9-3541 | 0.24 | 0.03 |
| siPCSK9-3542 | 0.19 | 0.06 |
| siPCSK9-3549 | 0.24 | 0.05 |
| siPCSK9-3553 | 0.16 | 0.03 |
| siPCSK9-3554 | 0.09 | 0.01 |
| siPCSK9-3556 | 0.10 | 0.01 |
| siPCSK9-3558 | 0.27 | 0.05 |
| siNC | 1.00 | 0.18 |
| Inclisiran | 0.16 | 0.05 |
| siPCSK9-3267 | 0.21 | 0.07 |
| siPCSK9-3580 | 0.22 | 0.10 |

The above results demonstrate that compared to the existing siRNAs (inclisiran, siPCSK9-3267, and siPCSK9-3580), most of the siRNAs of the present invention exhibit a stronger inhibitory effect on PCSK9 gene expression, and have the potential of more efficiently increasing LDL-R activity in vivo, decreasing the plasma LDL-C level, and reducing the risk of cardiovascular diseases.

### Example 3: Free Uptake Experiment on Cynomolgus Monkey Primary Hepatocytes

The sequences of siPCSK9-614, siPCSK9-2102, siPCSK9-3534, siPCSK9-3536, siPCSK9-3554 and siPCSK9-3556 were modified, and the modified sequences are shown in Table 5, wherein m represents 2′-OMe modification on the adjacent nucleotide to the left, f represents 2′-F modification on the adjacent nucleotide to the left, s represents phosphorothioate modification on the phosphate backbone, and L96 represents N-acetylgalactosamine (GalNAc).

TABLE 5
Modified sequences

| Name | Sense strand 5′-3′ | Antisense strand 5′-3′ |
|---|---|---|
| siPCSK9- | CmsCmsAmAmGmAm(Uf)Cm(Cf)Um(Gf)CmA | Ams(Af)s(Gf)(Af)Cm(Af)Um(Gf)Cm(Af)Gm |
| 614-m1-L96 | mUmGmUmCmUmUmCmCmL96 (SEQ ID | (Gf)Am(Uf)Cm(Uf)UmGmGmsUmsGm (SEQ ID |
| | NO: 73) | NO: 85) |
| siPCSK9- | CmsCmsAmAmGmAm(Uf)Cm(Cf)(Uf)(Gf)CmA | AmsAmsGm(Af)Cm(Af)(Uf)GmCmAmGm(Gf) |
| 614-m2-L96 | mUmGmUmCmUmUmCmCmL96 (SEQ ID | Am(Uf)CmUmUmGmGmsUmsGm (SEQ ID |
| | NO: 74) | NO: 86) |
| siPCSK9- | GmsUmsCmUmGmGm(Af)Am(Uf)Gm(Cf)AmA | Ums(Uf)s(Gf)(Af)Cm(Uf)Um(Uf)Gm(Cf)Am |
| 2102-m1- | mAmGmUmCmAmAmGmGmL96 (SEQ ID | (Uf)Um(Cf)Cm(Af)GmAmCmsCmsUm (SEQ ID |
| L96 | NO: 75) | NO: 87) |
| siPCSK9- | GmsUmsCmUmGmGm(Af)Am(Uf)(Gf)(Cf)Am | UmsUmsGm(Af)Cm(Uf)(Uf)UmGmCmAm(Uf) |
| 2102-m2- | AmAmGmUmCmAmAmGmGmL96 (SEQ ID | Um(Cf)CmAmGmAmCmsCmsUm (SEQ ID |
| L96 | NO: 76) | NO: 88) |
| siPCSK9- | CmsCmsUmGmUmUm(Uf)Um(Gf)Cm(Uf)UmU | Ams(Af)sGm(Uf)(Uf)(Af)Cm(Af)Am(Af)Am |
| 3534-m1- | mUmGmUmAmAmCmUmUmL96 (SEQ ID | (Gf)Cm(Af)Am(Af)Am(Cf)AmGmGmsUmsCm |
| L96 | NO: 77) | (SEQ ID NO: 89) |
| siPCSK9- | CmsCmsUmGmUmUm(Uf)Um(Gf)(Cf)(Uf)Um | Ams(Af)sGmUmUm(Af)Cm(Af)(Af)AmAmGm |
| 3534-m2- | UmUmGmUmAmAmCmUmUmL96 (SEQ ID | Cm(Af)Am(Af)AmCmAmGmGmsUmsCm (SEQ |
| L96 | NO: 78) | ID NO: 90) |
| siPCSK9- | UmsGmsUmUmUmUm(Gf)Cm(Uf)Um(Uf)UmG | Ums(Cf)sAm(Af)(Gf)(Uf)Um(Af)Cm(Af)Am |
| 3536-m1- | mUmAmAmCmUmUmGmAmL96 (SEQ ID | (Af)Am(Gf)Cm(Af)Am(Af)AmCmAmsGmsGm |
| L96 | NO: 79) | (SEQ ID NO: 91) |
| siPCSK9- | UmsGmsUmUmUmUm(Gf)Cm(Uf)(Uf)(Uf)Um | Ums(Cf)sAmAmGm(Uf)Um(Af)(Cf)AmAmAm |
| 3536-m2- | GmUmAmAmCmUmUmGmAmL96 (SEQ ID | Am(Gf)Cm(Af)AmAmAmCmAmsGmsGm (SEQ |
| L96 | NO: 80) | ID NO: 92) |
| siPCSK9- | UmsGmsAmAmGmAm(Uf)Am(Uf)Um(Uf)Am | Ams(Af)sCm(Cf)(Cf)(Af)Gm(Af)Am(Uf)Am |
| 3554-m1- | UmUmCmUmGmGmGmUmUmL96 (SEQ ID | (Af)Am(Uf)Am(Uf)Cm(Uf)UmCmAmsAmsGm |
| L96 | NO: 81) | (SEQ ID NO: 93) |
| siPCSK9- | UmsGmsAmAmGmAm(Uf)Am(Uf)(Uf)(Uf)Am | Ams(Af)sCmCmCm(Af)Gm(Af)(Af)UmAmAm |
| 3554-m2- | UmUmCmUmGmGmGmUmUmL96 (SEQ ID | Am(Uf)Am(Uf)CmUmUmCmAmsAmsGm (SEQ |
| L96 | NO: 82) | ID NO: 94) |
| siPCSK9- | AmsAmsGmAmUmAm(Uf)Um(Uf)Am(Uf)UmC | Ams(Af)sAm(Af)(Cf)(Cf)Cm(Af)Gm(Af)Am |
| 3556-m1- | mUmGmGmGmUmUmUmUmL96 (SEQ ID | (Uf)Am(Af)Am(Uf)Am(Uf)CmUmUmsCmsAm |
| L96 | NO: 83) | (SEQ ID NO: 95) |
| siPCSK9- | AmsAmsGmAmUmAm(Uf)Um(Uf)(Af)(Uf)Um | Ams(Af)sAmAmCm(Cf)Cm(Af)(Gf)AmAmUm |
| 3556-m2- | CmUmGmGmGmUmUmUmUmL96 (SEQ ID | Am(Af)Am(Uf)AmUmCmUmUmsCmsAm (SEQ |
| L96 | NO: 84) | ID NO: 96) |

The above modified siRNA was diluted with PBS, and siRNA solution was added to a 24-well cell culture plate with 50 μL/well. Cynomolgus monkey primary hepatocytes were provided by Milecell Biological Science & Technology Co., Ltd., and were resuscitated and resuspended with supporting culture medium, then added to the cell culture plate and mixed with the siRNA solution, with a total volume of 500 μL, a cell concentration of 2×10⁵ cells/well, and a siRNA working concentration of 500 nM. Inclisiran and GalNAc-1955 of the same concentration were used as controls, wherein GalNAc-1955 is an unrelated sequence, which is from the patent CN104854242B of Alnylam (sense strand: 5′-CmUmUmACmGCmUmGAGUmACmUmUmCmGAL96-3′ (SEQ ID NO: 97), antisense strand: 5′-UCGAAGUmACUCmAGCGUmAAGdTsdT-3′ (SEQ ID NO: 98)).

The culture plate was placed in an incubator at 37° C. with 5% CO₂ and incubated for 24 h, and then the cells were collected. RNA was extracted according to the method in Example 2, and the relative expression level of PCSK9 mRNA in cynomolgus monkeys was detected. The primers used are from patent CN104854242B, and have the following sequences:

cynoGAPDH-F:
(SEQ ID NO: 99)
GCATCCTGGGCTACACTGA;
cynoGAPDH-R:
(SEQ ID NO: 100)
TGGGTGTCGCTGTTGAAGTC;
cynoPCSK9-F:
(SEQ ID NO: 101)
ACGTGGCTGGCATTGCA;
cynoPCSK9-R:
(SEQ ID NO: 102)
AAGTGGATCAGTCTCTGCCTCAA.

The test results (see Table 6) show that most of the modified siPCSK9 had a stronger inhibitory effect on gene expression than Inclisiran in cynomolgus monkey primary hepatocytes, in which siPCSK9-614-m1-L96, siPCSK9-3534-m1-L96, siPCSK9-3536-m1-L96, siPCSK9-3556-m1-L96, and siPCSK9-3556-m2-L96 exhibited more than 60% inhibition.

TABLE 6
Inhibition of siPCSK9 modified sequences on genes
tested in cynomolgus monkey primary hepatocytes

| | |
|---|---|
| | Relative expression |
| Free uptake experiment on cynomolgus | level of PCSK9 |

| monkey primary hepatocytes | Mean | SD |
|---|---|---|
| siPCSK9-614-m1-L96 | 0.33 | 0.04 |
| siPCSK9-614-m2-L96 | 0.57 | 0.06 |
| siPCSK9-2102-m1-L96 | 0.61 | 0.15 |
| siPCSK9-2102-m2-L96 | 1.25 | 0.51 |
| siPCSK9-3534-m1-L96 | 0.31 | 0.12 |
| siPCSK9-3534-m2-L96 | 0.41 | 0.05 |
| siPCSK9-3536-m1-L96 | 0.38 | 0.03 |
| siPCSK9-3536-m2-L96 | 0.49 | 0.05 |
| siPCSK9-3554-m1-L96 | 0.47 | 0.03 |
| siPCSK9-3554-m2-L96 | 0.51 | 0.02 |
| siPCSK9-3556-m1-L96 | 0.35 | 0.04 |
| siPCSK9-3556-m2-L96 | 0.31 | 0.04 |
| GalNAc-1955 | 1.00 | 0.03 |
| Inclsiran | 0.61 | 0.05 |

### Example 4: In Vivo Animal Test for Inhibition Effect of siRNA on PCSK9 Protein

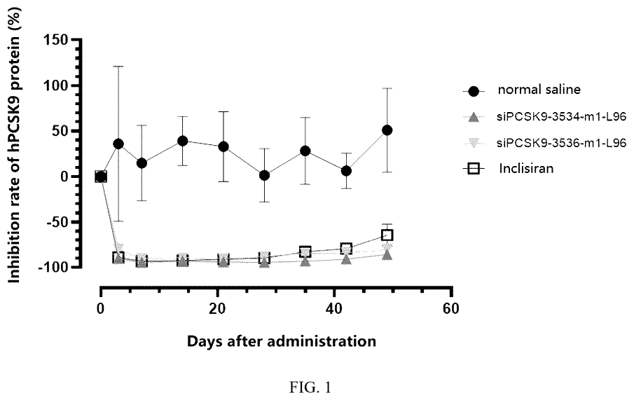

In order to evaluate the effect of the modified siPCSK9 in Example 3 on PCSK9 protein content in vivo, SPF grade B6-hPCSK9-UTR male mice (GemPharmatech Co., Ltd.) aged 6-8 weeks old were prepared and fed adaptively for 3-5 days. The mice were weighed on Day-3, and blood samples were collected 5 hours after eating. The plasma hPCSK9 protein level was detected by ELISA, and the mice were randomly divided into 7 groups with 6 mice in each group according to the protein level. The day of administration was defined as Day0. The test articles were siPCSK9-3534-m1-L96 and siPCSK9-3536-m1-L96, and the controls were normal saline and Inclisiran (Novartis). Mode of administration: subcutaneous injection; Frequency of administration: single administration; Dosage: 6 mg/kg; Volume of administration: 5 μL/g. Blood samples were collected at Day 0, 3, 7, 14, 21, 28, 35, 42 and 49, respectively. Blood samples were collected after fasting for 5 h. Plasma hPCSK9 protein levels were detected by ELISA. The results are shown in Table 7 and FIG. 1.

TABLE 7
Inhibition rate of siRNA on hPCSK9 protein in mice

| hPCSK9 inhibition rate | Day 7 | | Day 14 | | Day 28 | | Day 49 | |
|---|---|---|---|---|---|---|---|---|
| (%) | Mean | SD | Mean | SD | Mean | SD | Mean | SD |
| normal saline | 14.53% | 41.40% | 39.06% | 26.95% | 1.31% | 29.32% | 50.89% | 46.17% |
| siPCSK9-3534-m1-L96 | −94.89% | 1.80% | −93.36% | 5.01% | −94.69% | 1.36% | −86.05% | 3.92% |
| siPCSK9-3536-m1-L96 | −90.08% | 3.73% | −89.28% | 2.61% | −87.92% | 3.09% | −80.29% | 5.36% |
| Inclisiran | −93.29% | 1.53% | −92.74% | 0.94% | −89.94% | 2.56% | −64.51% | 12.26% |

As shown in Table 7, on day 49 after single administration, the protein inhibition rates of siPCSK9-3534-m1-L96 and siPCSK9-3536-m1-L96 were 86.05% and −80.29%, respectively, while the protein inhibition rate of Inclisiran was-64.51% (Table 7 and FIG. 1), indicating that the siRNA of the present invention can obviously inhibit the protein content of hPCSK9 in mouse plasma.

The above description is only for the purpose of illustrating the preferred examples of the present invention, and is not intended to limit the scope of the present invention. Any modifications, equivalents and the like made without departing from the spirit and principle of the present invention shall fall within the protection scope of the present invention.

1. An interfering RNA for inhibiting PCSK9 gene expression, comprising a nucleotide sequence set forth in any one or two or more of SEQ ID NOs: 1-40, 73-96.

2. The interfering RNA according to claim 1, wherein a sense strand of the interfering RNA is selected from any one or a combination of two or more of nucleotide sequences set forth in SEQ ID NOs: 1-20, 73-84, and an antisense strand of the interfering RNA is selected from any one or a combination of two or more of nucleotide sequences set forth in SEQ ID NOs: 21-40, 85-96.

3. The interfering RNA according to claim 2, comprising at least one of the following combinations: a combination of SEQ ID NO: 1 and SEQ ID NO: 21, a combination of SEQ ID NO: 2 and SEQ ID NO: 22, a combination of SEQ ID NO: 3 and SEQ ID NO: 23, a combination of SEQ ID NO: 4 and SEQ ID NO: 24, a combination of SEQ ID NO: 5 and SEQ ID NO: 25, a combination of SEQ ID NO: 6 and SEQ ID NO: 26, a combination of SEQ ID NO: 7 and SEQ ID NO: 27, a combination of SEQ ID NO: 8 and SEQ ID NO: 28, a combination of SEQ ID NO: 9 and SEQ ID NO: 29, a combination of SEQ ID NO: 10 and SEQ ID NO: 30, a combination of SEQ ID NO: 11 and SEQ ID NO: 31, a combination of SEQ ID NO: 12 and SEQ ID NO: 32, a combination of SEQ ID NO: 13 and SEQ ID NO: 33, a combination of SEQ ID NO: 14 and SEQ ID NO: 34, a combination of SEQ ID NO: 15 and SEQ ID NO: 35, a combination of SEQ ID NO: 16 and SEQ ID NO: 36, a combination of SEQ ID NO: 17 and SEQ ID NO: 37, a combination of SEQ ID NO: 18 and SEQ ID NO: 38, a combination of SEQ ID NO: 19 and SEQ ID NO: 39, a combination of SEQ ID NO: 20 and SEQ ID NO: 40, a combination of SEQ ID NO:73 and SEQ ID NO:85, a combination of SEQ ID NO: 74 and SEQ ID NO:86, a combination of SEQ ID NO:75 and SEQ ID NO:87, a combination of SEQ ID NO:76 and SEQ ID NO:88, a combination of SEQ ID NO:77 and SEQ ID NO:89, a combination of SEQ ID NO:78 and SEQ ID NO:90, a combination of SEQ ID NO:79 and SEQ ID NO: 91, a combination of SEQ ID NO:80 and SEQ ID NO:92, a combination of SEQ ID NO:81 and SEQ ID NO:93, a combination of SEQ ID NO:82 and SEQ ID NO:94, a combination of SEQ ID NO: 83 and SEQ ID NO:95, and a combination of SEQ ID NO:84 and SEQ ID NO:96; preferably, comprising a combination of SEQ ID NO:77 and SEQ ID NO:89, or a combination of SEQ ID NO:79 and SEQ ID NO:91.

4. The interfering RNA according to claim 1, wherein the 3′ ends of the sense strand and the antisense strand of the interfering RNA have overhangs of 0, 1, or 2 nucleotides.

5. The interfering RNA according to claim 1, further comprising at least one modified nucleotide, wherein the modification comprises a modification on a base, a modification on a sugar ring, and/or a modification on a phosphate backbone;
the modification on a base comprises pyrimidine modification at position 5, purine modification at position 8, pseudouridine modification, and/or 5-bromouracil substitution;
the modification on a sugar ring comprises 2′-hydroxyl modification, 2′-fluoro modification, 2′-deoxy modification, 2′-O-methyl modification, 2′-O-methoxyethyl modification, 2′-O-allyl modification, 2′-C-allyl modification, and locked nucleic acid modification;
the modification on a phosphate backbone comprises phosphorothioate modification and ligand modification.

6. The interfering RNA according to claim 5, wherein the modified nucleotide comprises nucleotides having inosine, queuosine, xanthine, 2′-methylribose, a non-natural phosphodiester bond, or a peptide.

7. The interfering RNA according to claim 5, wherein the ligand is selected from any one or a combination of two or more of cholesterol, biotin, a vitamin, a galactose derivative or analog, a lactose derivative or analog, N-acetylgalactosamine (GalNAc), an N-acetylgalactosamine derivative or analog, an N-acetylglucosamine derivative or analog, and a mannose 6-phosphate (M6P) derivative or analog.

8. The interfering RNA according to claim 7, wherein the GalNAc derivative or analog has a structural formula as follows:

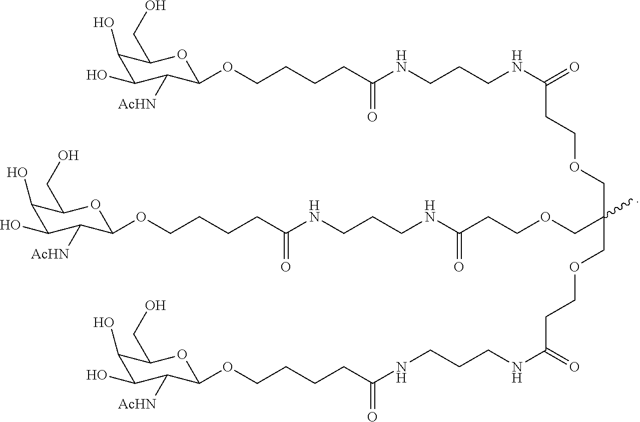 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

9. The interfering RNA according to claim 8, having a structural formula as follows: 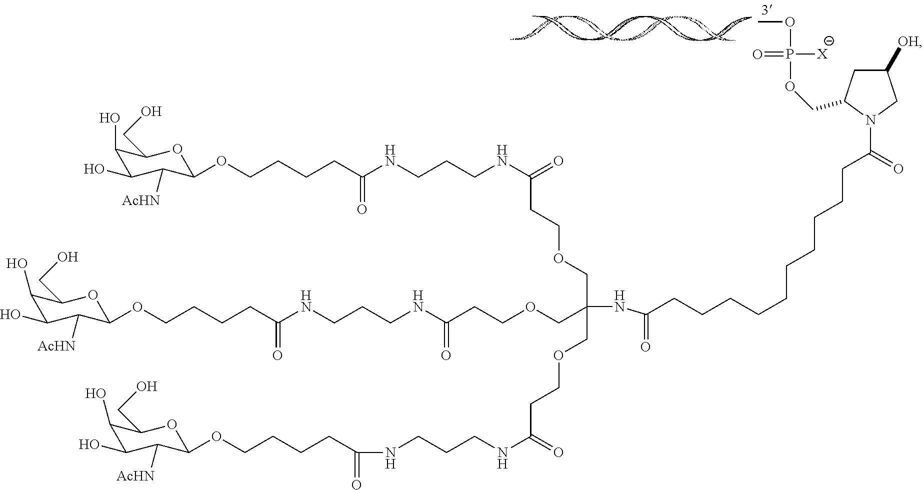
wherein X represents S or O.

10. A delivery system for an interfering RNA, comprising the interfering RNA according to claim 1 and a vector.

11. The delivery system according to claim 10, wherein the vector is a viral vector or a non-viral vector;
the viral vector comprises one or a combination of two or more of a lentivirus vector, a retrovirus vector, an adenovirus vector, an adeno-associated virus vector, a poxvirus vector, or a herpesvirus vector;
the non-viral vector comprises any one or a combination of two or more of a liposome, a lipid nanoparticle, a polymer, a polypeptide, an antibody, or an aptamer.

12. The delivery system according to claim 11, wherein the lipid nanoparticle or the liposome comprises one or a combination of two or more of a cationic lipid, a neutral lipid, a polyethylene glycol lipid, a steroidal lipid, or an anionic lipid.

13. The delivery system according to claim 12, wherein the cationic lipid comprises one or a combination of two or more of stearamide (SA), lauryltrimethylammonium bromide, hexadecyltrimethylammonium bromide, myristyltrimethylammonium bromide, dimethyldioctadecylammonium bromide (DDAB), [(4-hydroxybutyl) azanediyl] di(hexane-6,1-diyl)bis(2-hexyldecanoate) (ALC-0315), 1,2-dioleoyloxy-3-(trimethylammonium) propane (DOTAP), 1,2-di-(9Z-octadecenoyl)-3-trimethylammonium-propane and 1,2-dihexadecanoyl-3-trimethylammonium-propane, 3B-[N-(N′,N′-dimethylaminoethane)-carbamoyl] cholesterol (DC-cholesterol), dimethyldioctadecylammonium (DDA), 1,2-dimyristoyl-3-trimethylammonium propane (DMTAP), dipalmitoyl(C16: 0)trimethylammonium propane (DPTAP), distearoyltrimethylammonium propane (DSTAP), N-[1-(2,3-diallyloxy) propyl]-N,N,N-trimethylammonium chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium propane (DODAP), 1,2-dilinoleyloxy-3-dimethylaminopropane (DLinDMA), 1,2-ditetradecanoyl-3-dimethylammonium-propane, 1,2-dihexadecanoyl-3-dimethylammonium-propane and 1,2-dioctadecanoyl-3-dimethylammonium-propane, 1,2-dioleoyl-c-(4′-trimethylammonium)-butanoyl-sn-glycerol (DOTB), dioctadecylamidoalanyl spermine, SAINT-2, polycationic lipid 2,3-dioleoyloxy-N-[2 (spermine-carboxamido)ethyl]-N,N-dimethyl-1-propanaminium trifluoroacetate 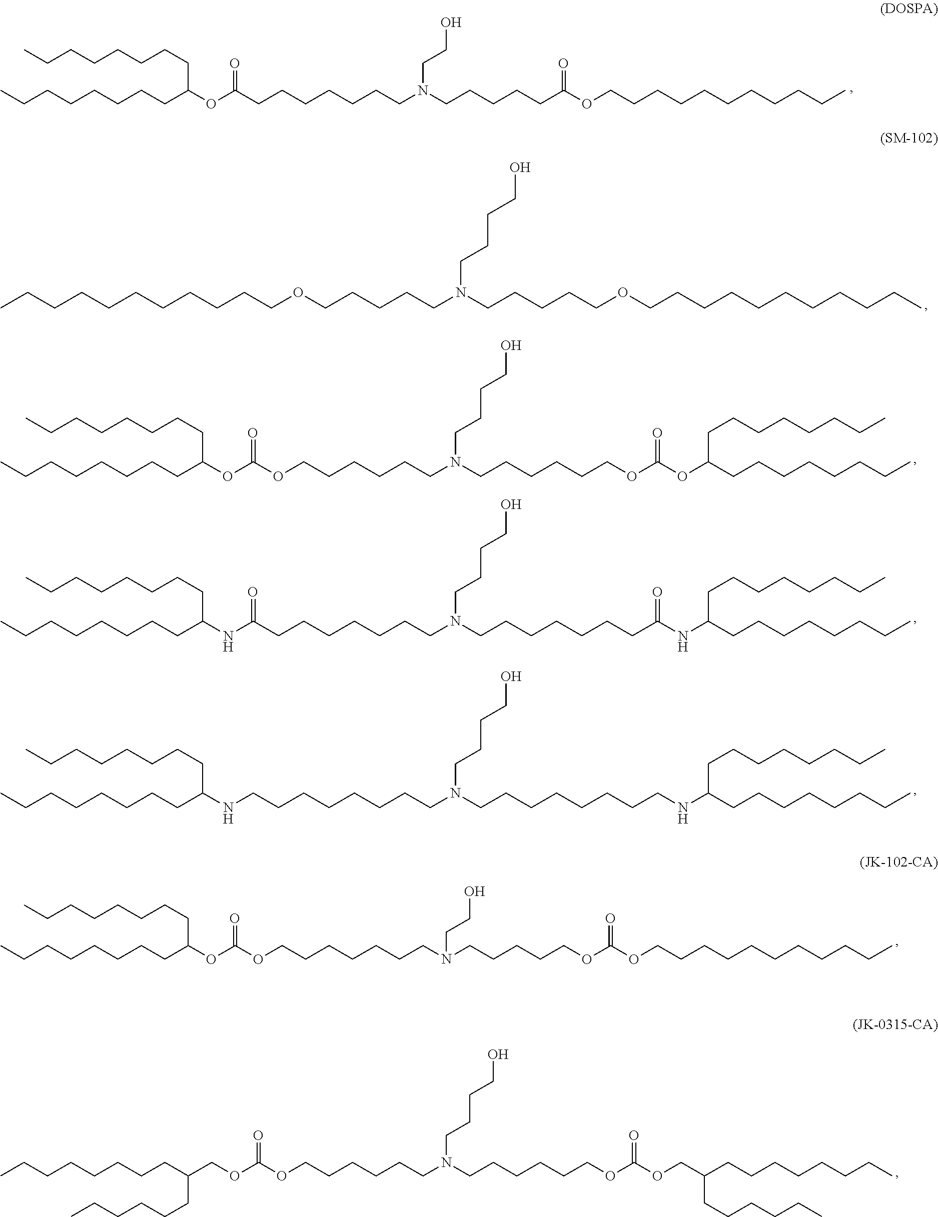
and Dlin-MC3-DMA; and/or, the neutral lipid comprises one or a combination of two or more of 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), 2-dioleoyl-sn-glycero-3-phospho-(1′-rac-glycerol) (DOPG), oleoyl phosphatidylcholine (POPC), 1-palmitoyl-2-oleoyl phosphatidylethanolamine (POPE), or distearoylphosphatidylethanolamine (DSPE).

14. The delivery system according to claim 12, wherein the polyethylene glycol lipid comprises one or a combination of two or more of 2-[(polyethylene glycol)-2000]-N,N-ditetradecylacetamide (ALC-0159), 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disterol glycerol (PEG-DSG), PEG-dipalmitoyl, PEG-dioleyl, PEG-distearyl, PEG-diacylglycerol amide (PEG-DAG), PEG-dipalmitoyl phosphatidylethanolamine (PEG-DPPE) or PEG-1,2-dimyristoyloxypropyl-3-amine (PEG-c-DMA), 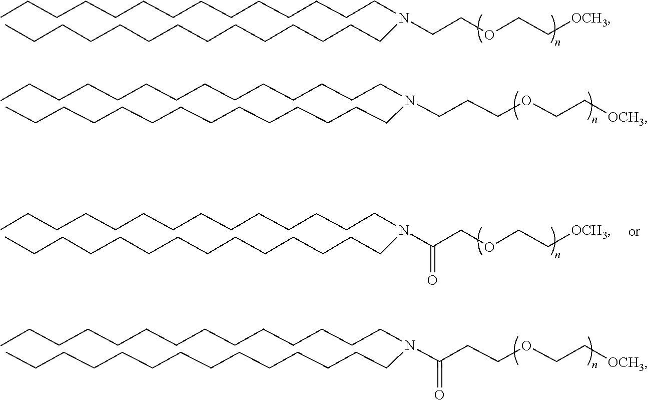
wherein n is selected from integers of 20-300; or,
the polyethylene glycol lipid is a polyethylene glycol lipid with a single molecular weight, and the polyethylene glycol lipid is selected from:

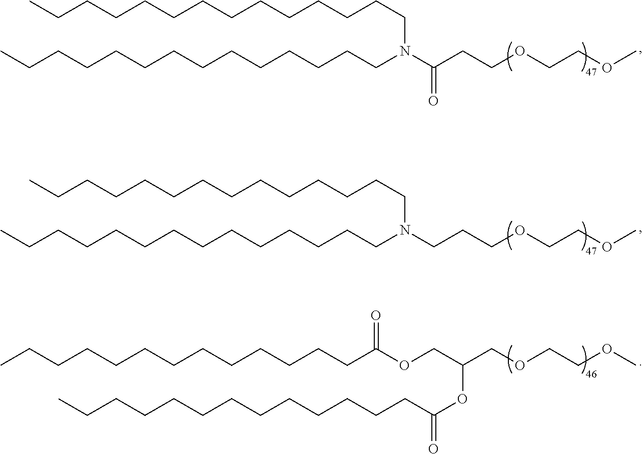

15. The delivery system according to claim 12, wherein the cationic lipid is a steroid-cationic lipid compound,
and the compound has a structure of:

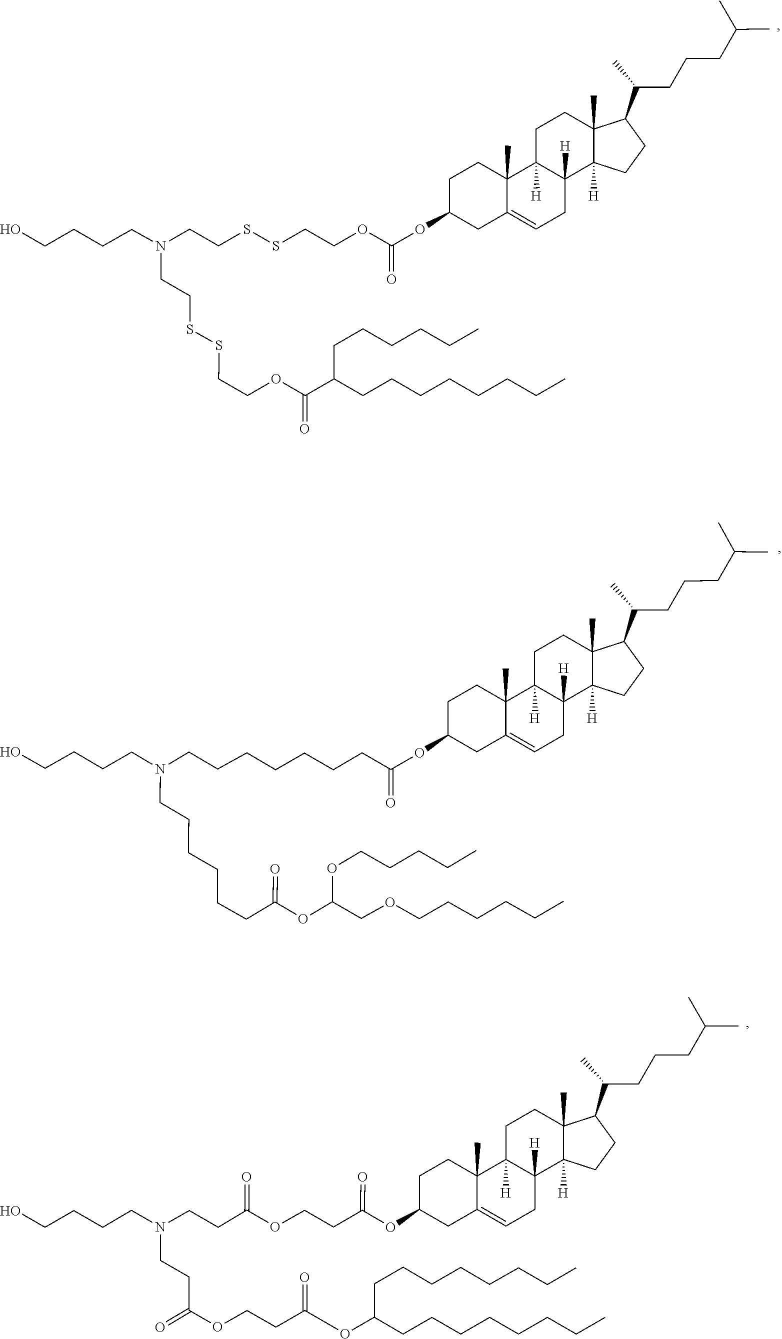

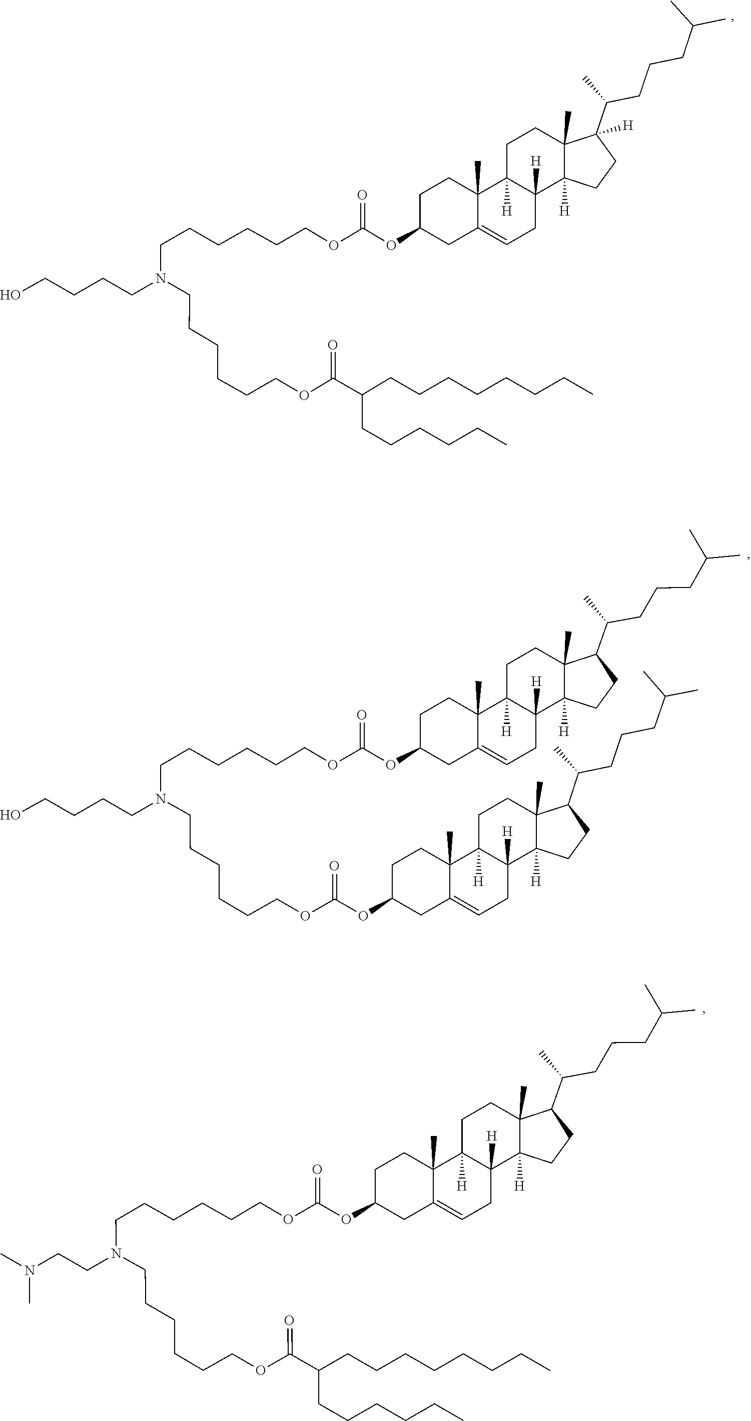

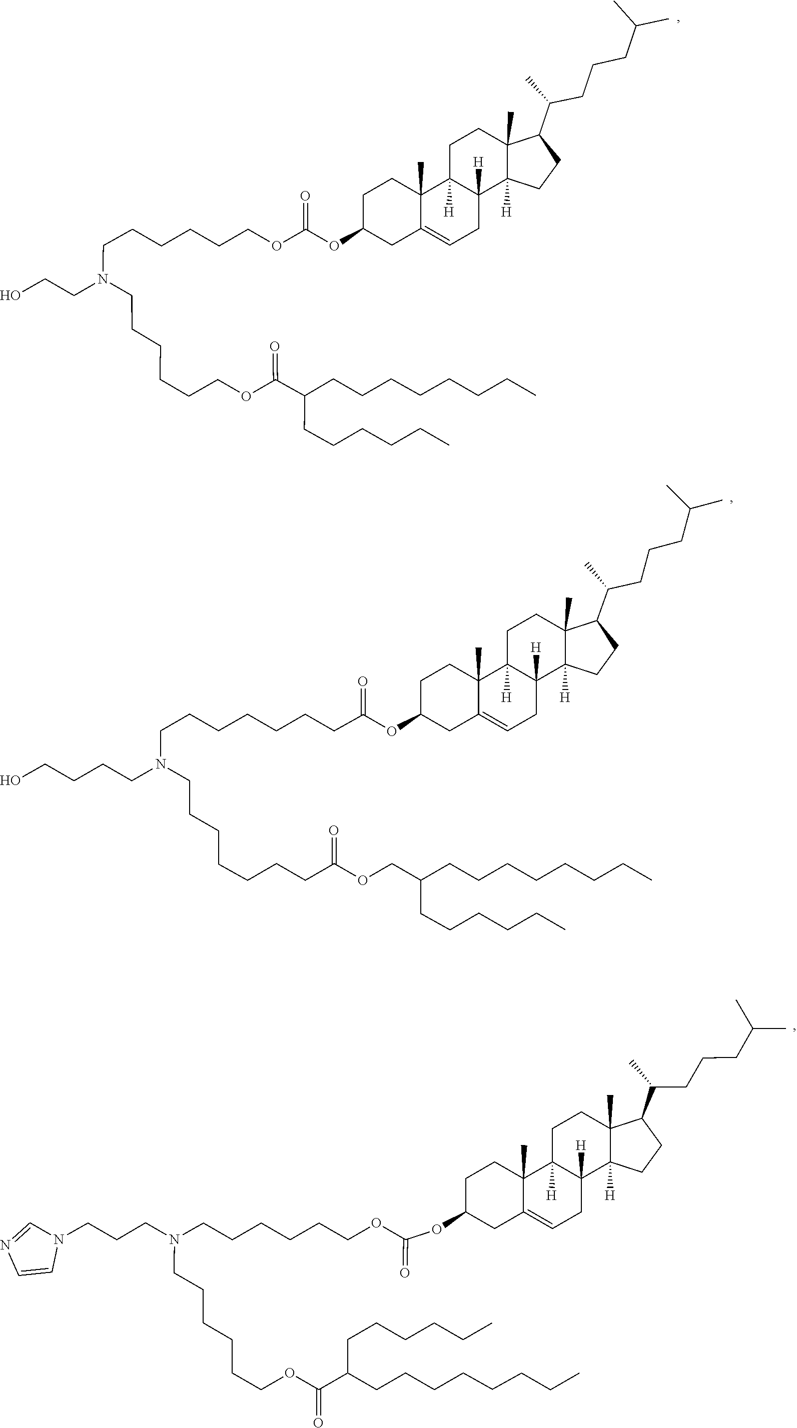

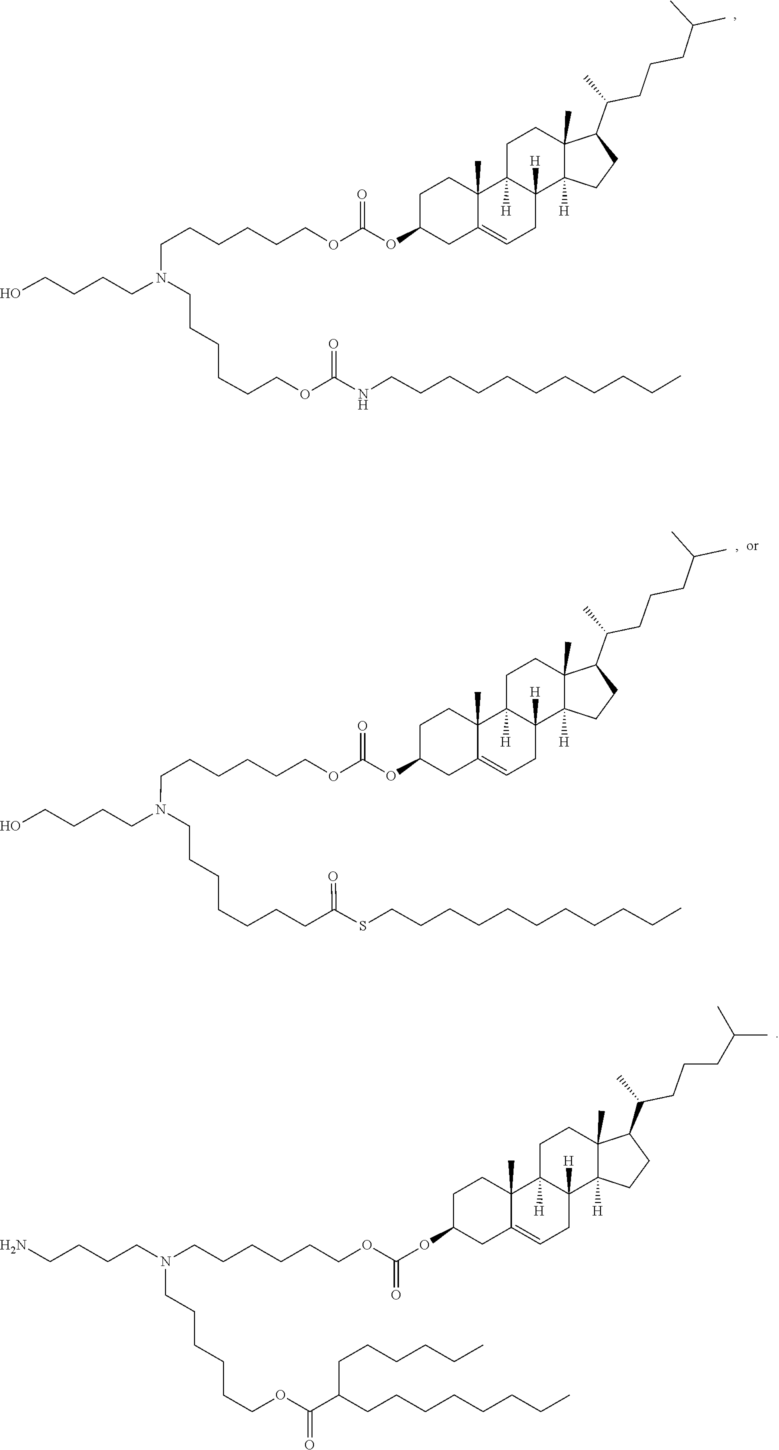

16. The delivery system according to claim 12, wherein the anionic liposome comprises one or a combination of two or more of dioleoyl phosphatidylglycerol or dioleoyl phosphatidylethanolamine; and/or,
the steroidal lipid comprises one or a combination of two or more of avenasterol, β-sitosterol, brassicasterol, ergocalciferol, campesterol, cholestanol, cholesterol, coprostanol, dehydrocholesterol, desmosterol, dihydroergocalciferol, dihydrocholesterol, dihydroergosterol, dinosterol, epicholesterol, ergosterol, fucosterol, hexahydrolumisterol, hydroxycholesterol, lanosterol, lumisterol, saringosterol, sitostanol, sitosterol, stigmastanol, stigmasterol, cholic acid, glycocholic acid, taurocholic acid, deoxycholic acid, or lithocholic acid.

17. A medicament or kit, comprising the interfering RNA according to claim 1.

18. Use of the interfering RNA according to claim 1
(1) in inhibiting PCSK9 gene expression;
(2) in reducing the concentration of low-density lipoprotein and/or low-density lipoprotein cholesterol in a serum;
(3) in preventing and/or treating a PCSK9 gene-mediated disease; or
(4) in alleviating a symptom of a PCSK9 gene-mediated disease.

19. The use according to claim 18, wherein the PCSK9 gene-mediated disease is a cardiovascular disease or a neoplastic disease.

20. The use according to claim 19, wherein the cardiovascular disease is selected from: hyperlipidemia, hypercholesterolemia, non-familial hypercholesterolemia, polygenic hypercholesterolemia, familial hypercholesterolemia, homozygous familial hypercholesterolemia, heterozygous familial hypercholesterolemia, and mixed dyslipidemia in mammals;
the neoplastic disease is PCSK9-associated melanoma or metastatic liver cancer.


SEQUENCE LISTING (102 sequences)

SEQ ID NO: 1 - RNA, 21 nt, synthetic construct
  source 1..21: mol_type = other RNA
cctacgtggtggtgctgaagg

SEQ ID NO: 2 - RNA, 21 nt, synthetic construct
  source 1..21: mol_type = other RNA
ccaagatcctgcatgtcttcc

SEQ ID NO: 3 - RNA, 21 nt, synthetic construct
  source 1..21: mol_type = other RNA
ggttagcggcaccctcatagg

SEQ ID NO: 4 - RNA, 21 nt, synthetic construct
  source 1..21: mol_type = other RNA
ggtacagccgcgtcctcaacg

SEQ ID NO: 5 - RNA, 21 nt, synthetic construct
  source 1..21: mol_type = other RNA
gcacctgctttgtgtcacaga

SEQ ID NO: 6 - RNA, 21 nt, synthetic construct
  source 1..21: mol_type = other RNA
gtctggaatgcaaagtcaagg

SEQ ID NO: 7 - RNA, 21 nt, synthetic construct
  source 1..21: mol_type = other RNA
cctgattaatggaggcttagc

SEQ ID NO: 8 - RNA, 21 nt, synthetic construct
  source 1..21: mol_type = other RNA
ggcagtgtgcagtggtgcatg

SEQ ID NO: 9 - RNA, 21 nt, synthetic construct
  source 1..21: mol_type = other RNA
cctacttcacagaggaagaaa

SEQ ID NO: 10 - RNA, 21 nt, synthetic construct
  source 1..21: mol_type = other RNA
cagaggaagaaacctggaacc

SEQ ID NO: 11 - RNA, 21 nt, synthetic construct
  source 1..21: mol_type = other RNA
cctgttttgcttttgtaactt

SEQ ID NO: 12 - RNA, 21 nt, synthetic construct
  source 1..21: mol_type = other RNA
tgttttgcttttgtaacttga

SEQ ID NO: 13 - RNA, 21 nt, synthetic construct
  source 1..21: mol_type = other RNA
tttgcttttgtaacttgaaga

SEQ ID NO: 14 - RNA, 21 nt, synthetic construct
  source 1..21: mol_type = other RNA
tgcttttgtaacttgaagata

SEQ ID NO: 15 - RNA, 21 nt, synthetic construct
  source 1..21: mol_type = other RNA
gcttttgtaacttgaagatat

SEQ ID NO: 16 - RNA, 21 nt, synthetic construct
  source 1..21: mol_type = other RNA
taacttgaagatatttattct

SEQ ID NO: 17 - RNA, 21 nt, synthetic construct
  source 1..21: mol_type = other RNA
ttgaagatatttattctgggt

SEQ ID NO: 18 - RNA, 21 nt, synthetic construct
  source 1..21: mol_type = other RNA
tgaagatatttattctgggtt

SEQ ID NO: 19 - RNA, 21 nt, synthetic construct
  source 1..21: mol_type = other RNA
aagatatttattctgggtttt

SEQ ID NO: 20 - RNA, 21 nt, synthetic construct
  source 1..21: mol_type = other RNA
gatatttattctgggttttgt

SEQ ID NO: 21 - RNA, 21 nt, synthetic construct
  source 1..21: mol_type = other RNA
ttcagcaccaccacgtaggtg

SEQ ID NO: 22 - RNA, 21 nt, synthetic construct
  source 1..21: mol_type = other RNA
aagacatgcaggatcttggtg

SEQ ID NO: 23 - RNA, 21 nt, synthetic construct
  source 1..21: mol_type = other RNA
tatgagggtgccgctaaccgt

SEQ ID NO: 24 - RNA, 21 nt, synthetic construct
  source 1..21: mol_type = other RNA
ttgaggacgcggctgtaccca

SEQ ID NO: 25 - RNA, 21 nt, synthetic construct
  source 1..21: mol_type = other RNA
tgtgacacaaagcaggtgctg

SEQ ID NO: 26 - RNA, 21 nt, synthetic construct
  source 1..21: mol_type = other RNA
ttgactttgcattccagacct

SEQ ID NO: 27 - RNA, 21 nt, synthetic construct
  source 1..21: mol_type = other RNA
taagcctccattaatcaggga

SEQ ID NO: 28 - RNA, 21 nt, synthetic construct
  source 1..21: mol_type = other RNA
tgcaccactgcacactgccga

SEQ ID NO: 29 - RNA, 21 nt, synthetic construct
  source 1..21: mol_type = other RNA
tcttcctctgtgaagtagggg

SEQ ID NO: 30 - RNA, 21 nt, synthetic construct
  source 1..21: mol_type = other RNA
ttccaggtttcttcctctgtg

SEQ ID NO: 31 - RNA, 23 nt, synthetic construct
  source 1..23: mol_type = other RNA
aagttacaaaagcaaaacaggtc

SEQ ID NO: 32 - RNA, 23 nt, synthetic construct
  source 1..23: mol_type = other RNA
tcaagttacaaaagcaaaacagg

SEQ ID NO: 33 - RNA, 23 nt, synthetic construct
  source 1..23: mol_type = other RNA
tcttcaagttacaaaagcaaaac

SEQ ID NO: 34 - RNA, 23 nt, synthetic construct
  source 1..23: mol_type = other RNA
tatcttcaagttacaaaagcaaa

SEQ ID NO: 35 - RNA, 23 nt, synthetic construct
  source 1..23: mol_type = other RNA
atatcttcaagttacaaaagcaa

SEQ ID NO: 36 - RNA, 23 nt, synthetic construct
  source 1..23: mol_type = other RNA
agaataaatatcttcaagttaca

SEQ ID NO: 37 - RNA, 23 nt, synthetic construct
  source 1..23: mol_type = other RNA
acccagaataaatatcttcaagt

SEQ ID NO: 38 - RNA, 23 nt, synthetic construct
  source 1..23: mol_type = other RNA
aacccagaataaatatcttcaag

SEQ ID NO: 39 - RNA, 23 nt, synthetic construct
  source 1..23: mol_type = other RNA
aaaacccagaataaatatcttca

SEQ ID NO: 40 - RNA, 23 nt, synthetic construct
  source 1..23: mol_type = other RNA
acaaaacccagaataaatatctt

SEQ ID NO: 41 - DNA, 23 bp, synthetic construct
  source 1..23: mol_type = other DNA
cacctacgtggtggtgctgaagg

SEQ ID NO: 42 - DNA, 23 bp, synthetic construct
  source 1..23: mol_type = other DNA
caccaagatcctgcatgtcttcc

SEQ ID NO: 43 - DNA, 23 bp, synthetic construct
  source 1..23: mol_type = other DNA
acggttagcggcaccctcatagg

SEQ ID NO: 44 - DNA, 23 bp, synthetic construct
  source 1..23: mol_type = other DNA
tgggtacagccgcgtcctcaacg

SEQ ID NO: 45 - DNA, 23 bp, synthetic construct
  source 1..23: mol_type = other DNA
cagcacctgctttgtgtcacaga

SEQ ID NO: 46 - DNA, 23 bp, synthetic construct
  source 1..23: mol_type = other DNA
aggtctggaatgcaaagtcaagg

SEQ ID NO: 47 - DNA, 23 bp, synthetic construct
  source 1..23: mol_type = other DNA
tccctgattaatggaggcttagc

SEQ ID NO: 48 - DNA, 23 bp, synthetic construct
  source 1..23: mol_type = other DNA
tcggcagtgtgcagtggtgcatg

SEQ ID NO: 49 - DNA, 23 bp, synthetic construct
  source 1..23: mol_type = other DNA
cccctacttcacagaggaagaaa

SEQ ID NO: 50 - DNA, 23 bp, synthetic construct
  source 1..23: mol_type = other DNA
cacagaggaagaaacctggaacc

SEQ ID NO: 51 - DNA, 23 bp, synthetic construct
  source 1..23: mol_type = other DNA
gacctgttttgcttttgtaactt

SEQ ID NO: 52 - DNA, 23 bp, synthetic construct
  source 1..23: mol_type = other DNA
cctgttttgcttttgtaacttga

SEQ ID NO: 53 - DNA, 23 bp, synthetic construct
  source 1..23: mol_type = other DNA
gttttgcttttgtaacttgaaga

SEQ ID NO: 54 - DNA, 23 bp, synthetic construct
  source 1..23: mol_type = other DNA
tttgcttttgtaacttgaagata

SEQ ID NO: 55 - DNA, 23 bp, synthetic construct
  source 1..23: mol_type = other DNA
ttgcttttgtaacttgaagatat

SEQ ID NO: 56 - DNA, 23 bp, synthetic construct
  source 1..23: mol_type = other DNA
tgtaacttgaagatatttattct

SEQ ID NO: 57 - DNA, 23 bp, synthetic construct
  source 1..23: mol_type = other DNA
acttgaagatatttattctgggt

SEQ ID NO: 58 - DNA, 23 bp, synthetic construct
  source 1..23: mol_type = other DNA
cttgaagatatttattctgggtt

SEQ ID NO: 59 - DNA, 23 bp, synthetic construct
  source 1..23: mol_type = other DNA
tgaagatatttattctgggtttt

SEQ ID NO: 60 - DNA, 23 bp, synthetic construct
  source 1..23: mol_type = other DNA
aagatatttattctgggttttgt

SEQ ID NO: 61 - RNA, 21 nt, synthetic construct
  source 1..21: mol_type = other RNA
ttctccgaacgtgtcacgttt

SEQ ID NO: 62 - RNA, 21 nt, synthetic construct
  source 1..21: mol_type = other RNA
acgtgacacgttcggagaatt

SEQ ID NO: 63 - RNA, 21 nt, synthetic construct
  source 1..21: mol_type = other RNA
  modified_base 1..6: mod_base = OTHER; note = 2-O-(2-methoxyethyl)
  modified_base 8: mod_base = OTHER; note = 2-O-(2-methoxyethyl)
  modified_base 10: mod_base = OTHER; note = 2-O-(2-methoxyethyl)
  modified_base 12..21: mod_base = OTHER; note = 2-O-(2-methoxyethyl)
  modified_base 1..2: mod_base = OTHER; note = phosphothionate
  modified_base 7: mod_base = OTHER; note = 2'-Fluoro
  modified_base 9: mod_base = OTHER; note = 2'-Fluoro
  modified_base 11: mod_base = OTHER; note = Thymine
ctagacctgttttgcttttgt

SEQ ID NO: 64 - RNA, 23 nt, synthetic construct
  source 1..23: mol_type = other RNA
  modified_base 1: mod_base = OTHER; note = 2-O-(2-methoxyethyl)
  modified_base 3: mod_base = OTHER; note = 2-O-(2-methoxyethyl)
  modified_base 7: mod_base = OTHER; note = 2-O-(2-methoxyethyl)
  modified_base 9: mod_base = OTHER; note = 2-O-(2-methoxyethyl)
  modified_base 11: mod_base = OTHER; note = 2-O-(2-methoxyethyl)
  modified_base 13: mod_base = OTHER; note = 2-O-(2-methoxyethyl)
  modified_base 15: mod_base = OTHER; note = 2-O-(2-methoxyethyl)
  modified_base 17: mod_base = OTHER; note = 2-O-(2-methoxyethyl)
  modified_base 19..23: mod_base = OTHER; note = 2-O-(2-methoxyethyl)
  modified_base 2: mod_base = OTHER; note = 2'-Fluoro
  modified_base 4..6: mod_base = OTHER; note = 2'-Fluoro
  modified_base 8: mod_base = OTHER; note = 2'-Fluoro
  modified_base 10: mod_base = OTHER; note = 2'-Fluoro
  modified_base 12: mod_base = OTHER; note = 2'-Fluoro
  modified_base 14: mod_base = OTHER; note = 2'-Fluoro
  modified_base 16: mod_base = OTHER; note = 2'-Fluoro
  modified_base 18: mod_base = OTHER; note = 2'-Fluoro
  modified_base 1..2: mod_base = OTHER; note = phosphothionate
  modified_base 21..22: mod_base = OTHER; note = phosphothionate
acaaaagcaaaacaggtctagaa

SEQ ID NO: 65 - RNA, 21 nt, synthetic construct
  source 1..21: mol_type = other RNA
catttttacgggtaacagtga

SEQ ID NO: 66 - RNA, 21 nt, synthetic construct
  source 1..21: mol_type = other RNA
actgttacccgtaaaaatgag

SEQ ID NO: 67 - RNA, 21 nt, synthetic construct
  source 1..21: mol_type = other RNA
gcatttttattaatatggtga

SEQ ID NO: 68 - RNA, 21 nt, synthetic construct
  source 1..21: mol_type = other RNA
accatattaataaaaatgcta

SEQ ID NO: 69 - DNA, 20 bp, synthetic construct
  source 1..20: mol_type = other DNA
tctgacttcaacagcgacac

SEQ ID NO: 70 - DNA, 20 bp, synthetic construct
  source 1..20: mol_type = other DNA
gccaaattcgttgtcatacc

SEQ ID NO: 71 - DNA, 20 bp, synthetic construct
  source 1..20: mol_type = other DNA
cacagagtgggacatcacag

SEQ ID NO: 72 - DNA, 21 bp, synthetic construct
  source 1..21: mol_type = other DNA
tttggcagagaagtggatcag

SEQ ID NO: 73 - RNA, 21 nt, synthetic construct
  source 1..21: mol_type = other RNA
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
  modified_base 1..2: mod_base = OTHER; note = phosphothionate
ccaagatcctgcatgtcttcc

SEQ ID NO: 74 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
ccaagatcctgcatgtcttcc

SEQ ID NO: 75 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
gtctggaatgcaaagtcaagg

SEQ ID NO: 76 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
gtctggaatgcaaagtcaagg

SEQ ID NO: 77 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
cctgttttgcttttgtaactt

SEQ ID NO: 78 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
cctgttttgcttttgtaactt

SEQ ID NO: 79 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
tgttttgcttttgtaacttga

SEQ ID NO: 80 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
tgttttgcttttgtaacttga

SEQ ID NO: 81 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
tgaagatatttattctgggtt

SEQ ID NO: 82 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
tgaagatatttattctgggtt

SEQ ID NO: 83 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9: mod_base = OTHER; note = 2'-Fluoro
  modified_base 10: mod_base = OTHER; note = 2-O-(2-methoxyethyl)
  modified_base 11: mod_base = OTHER; note = 2'-Fluoro
  modified_base 12..21: mod_base = OTHER; note = 2-O-(2-methoxyethyl)
aagatatttattctgggtttt

SEQ ID NO: 84 - RNA, 21 nt, synthetic construct
  source 1..21: mol_type = other RNA
  modified_base 1..2: mod_base = OTHER; note = phosphothionate
  modified_base 1..6: mod_base = OTHER; note = 2-O-(2-methoxyethyl)
  modified_base 7: mod_base = OTHER; note = 2'-Fluoro
  modified_base 8: mod_base = OTHER; note = 2-O-(2-methoxyethyl)
  modified_base 9..11: mod_base = OTHER; note = 2'-Fluoro
  modified_base 12..21: mod_base = OTHER; note = 2-O-(2-methoxyethyl)
aagatatttattctgggtttt

SEQ ID NO: 85 - RNA, 21 nt, synthetic construct
  source 1..21: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3..4: mod_base = OTHER; note = 2'-Fluoro
  modified_base 5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17..21: mod_base = OTHER; note = 2-O-(2-methoxyethyl)
  modified_base 19..20: mod_base = OTHER; note = phosphothionate
aagacatgcaggatcttggtg

SEQ ID NO: 86 - RNA, 21 nt, synthetic construct
  source 1..21: mol_type = other RNA
  modified_base 1..2: mod_base = OTHER; note = phosphothionate
  modified_base 1..3: mod_base = OTHER; note = 2-O-(2-methoxyethyl)
  modified_base 4: mod_base = OTHER; note = 2'-Fluoro
  modified_base 5: mod_base = OTHER; note = 2-O-(2-methoxyethyl)
  modified_base 6..7: mod_base = OTHER; note = 2'-Fluoro
  modified_base 8..11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15..21: mod_base = OTHER; note = 2-O-(2-methoxyethyl)
  modified_base 19..20: mod_base = OTHER; note = phosphothionate
aagacatgcaggatcttggtg

SEQ ID NO: 87 - RNA, 21 nt, synthetic construct
  source 1..21: mol_type = other RNA
  modified_base 1..2: mod_base = OTHER; note = phosphothionate
  modified_base 2: mod_base = OTHER; note = 2'-Fluoro
  modified_base 3..4: mod_base = OTHER; note = 2'-Fluoro
  modified_base 5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 19..20: mod_base = OTHER; note = phosphothionate
  modified_base 1: mod_base = OTHER; note = 2-O-(2-methoxyethyl)
  modified_base 17..21: mod_base = OTHER; note = 2-O-(2-methoxyethyl)
ttgactttgcattccagacct

SEQ ID NO: 88 - RNA, 21 nt, synthetic construct
  source 1..21: mol_type = other RNA
  modified_base 1..2: mod_base = OTHER; note = phosphothionate
  modified_base 1..3: mod_base = OTHER; note = 2-O-(2-methoxyethyl)
  modified_base 4: mod_base = OTHER; note = 2'-Fluoro
  modified_base 5: mod_base = OTHER; note = 2-O-(2-methoxyethyl)
  modified_base 6..7: mod_base = OTHER; note = 2'-Fluoro
  modified_base 8..11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15..21: mod_base = OTHER; note = 2-O-(2-methoxyethyl)
  modified_base 19..20: mod_base = OTHER; note = phosphothionate
ttgactttgcattccagacct

SEQ ID NO: 89 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3: mod_base = OTHER; note = 2-O-(2-methoxyethyl)
  modified_base 4..6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17: mod_base = OTHER; note = 2-O-(2-methoxyethyl)
  modified_base 18: mod_base = OTHER; note = 2'-Fluoro
  modified_base 19..23: mod_base = OTHER; note = 2-O-(2-methoxyethyl)
  modified_base 21..22: mod_base = OTHER; note = phosphothionate
aagttacaaaagcaaaacaggtc

SEQ ID NO: 90 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3..5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8..9: mod_base = OTHER; note = 2'-Fluoro
  modified_base 10..13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17..23: mod_base = OTHER; note = 2-O-(2-methoxyethyl)
  modified_base 21..22: mod_base = OTHER; note = phosphothionate
aagttacaaaagcaaaacaggtc

SEQ ID NO: 91 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3: mod_base = OTHER; note = 2-O-(2-methoxyethyl)
  modified_base 4..6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17: mod_base = OTHER; note = 2-O-(2-methoxyethyl)
  modified_base 18: mod_base = OTHER; note = 2'-Fluoro
  modified_base 19..23: mod_base = OTHER; note = 2-O-(2-methoxyethyl)
  modified_base 21..22: mod_base = OTHER; note = phosphothionate
tcaagttacaaaagcaaaacagg

SEQ ID NO: 92 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3..5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8..9: mod_base = OTHER; note = 2'-Fluoro
  modified_base 10..13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17..23: mod_base = OTHER; note = 2-O-(2-methoxyethyl)
  modified_base 21..22: mod_base = OTHER; note = phosphothionate
tcaagttacaaaagcaaaacagg

SEQ ID NO: 93 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3: mod_base = OTHER; note = 2-O-(2-methoxyethyl)
  modified_base 4..6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17: mod_base = OTHER; note = 2-O-(2-methoxyethyl)
  modified_base 18: mod_base = OTHER; note = 2'-Fluoro
  modified_base 19..23: mod_base = OTHER; note = 2-O-(2-methoxyethyl)
  modified_base 21..22: mod_base = OTHER; note = phosphothionate
aacccagaataaatatcttcaag

SEQ ID NO: 94 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3..5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8..9: mod_base = OTHER; note = 2'-Fluoro
  modified_base 10..13: mod_base = OTHER; note = 2-O-(2-methoxyethyl)
  modified_base 14: mod_base = OTHER; note = 2'-Fluoro
  modified_base 15: mod_base = OTHER; note = 2'-Fluoro
  modified_base 16: mod_base = OTHER; note = 2'-Fluoro
  modified_base 17..23: mod_base = OTHER; note = 2-O-(2-methoxyethyl)
  modified_base 21..22: mod_base = OTHER; note = phosphothionate
aacccagaataaatatcttcaag

SEQ ID NO: 95 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3: mod_base = OTHER; note = 2-O-(2-methoxyethyl)
  modified_base 4..6: mod_base = OTHER; note = 2'-Fluoro
  modified_base 7: mod_base = OTHER; note = 2-O-(2-methoxyethyl)
  modified_base 8: mod_base = OTHER; note = 2'-Fluoro
  modified_base 9: mod_base = OTHER; note = 2-O-(2-methoxyethyl)
  modified_base 10: mod_base = OTHER; note = 2'-Fluoro
  modified_base 11: mod_base = OTHER; note = 2-O-(2-methoxyethyl)
  modified_base 12: mod_base = OTHER; note = 2'-Fluoro
  modified_base 13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17: mod_base = OTHER; note = 2-O-(2-methoxyethyl)
  modified_base 18: mod_base = OTHER; note = 2'-Fluoro
  modified_base 19..23: mod_base = OTHER; note = 2-O-(2-methoxyethyl)
  modified_base 21..22: mod_base = OTHER; note = phosphothionate
aaaacccagaataaatatcttca

SEQ ID NO: 96 - RNA, 23 nt, synthetic construct
  source 1..23: mol_type = other RNA
  modified_base 1..2: mod_base = OTHER; note = phosphothionate
  modified_base 1: mod_base = OTHER; note = 2-O-(2-methoxyethyl)
  modified_base 2: mod_base = OTHER; note = 2'-Fluoro
  modified_base 3..5: mod_base = OTHER; note = 2-O-(2-methoxyethyl)
  modified_base 6: mod_base = OTHER; note = 2'-Fluoro
  modified_base 7: mod_base = OTHER; note = 2-O-(2-methoxyethyl)
  modified_base 8..9: mod_base = OTHER; note = 2'-Fluoro
  modified_base 10..13: mod_base = OTHER; note = 2-O-(2-methoxyethyl)
  modified_base 14: mod_base = OTHER; note = 2'-Fluoro
  modified_base 15: mod_base = OTHER; note = 2-O-(2-methoxyethyl)
  modified_base 16: mod_base = OTHER; note = 2'-Fluoro
  modified_base 17..23: mod_base = OTHER; note = 2-O-(2-methoxyethyl)
  modified_base 21..22: mod_base = OTHER; note = phosphothionate
aaaacccagaataaatatcttca

SEQ ID NO: 97 - RNA, 19 nt, synthetic construct
  source 1..19: mol_type = other RNA
  modified_base 1..3: mod_base = OTHER; note = 2-O-(2-methoxyethyl)
  modified_base 5: mod_base = OTHER; note = 2-O-(2-methoxyethyl)
  modified_base 7..8: mod_base = OTHER; note = 2-O-(2-methoxyethyl)
  modified_base 12: mod_base = OTHER; note = 2-O-(2-methoxyethyl)
  modified_base 14..17: mod_base = OTHER; note = 2-O-(2-methoxyethyl)
cttacgctgagtacttcga

SEQ ID NO: 98 - RNA, 21 nt, synthetic construct
  source 1..21: mol_type = other RNA
  modified_base 7: mod_base = OTHER; note = 2-O-(2-methoxyethyl)
  modified_base 11: mod_base = OTHER; note = 2-O-(2-methoxyethyl)
  modified_base 16: mod_base = OTHER; note = 2-O-(2-methoxyethyl)
  modified_base 20..21: mod_base = OTHER; note = Thymine
tcgaagtactcagcgtaagtt

SEQ ID NO: 99 - DNA, 19 bp, synthetic construct
  source 1..19: mol_type = other DNA
gcatcctgggctacactga

SEQ ID NO: 100 - DNA, 20 bp, synthetic construct
  source 1..20: mol_type = other DNA
tgggtgtcgctgttgaagtc

SEQ ID NO: 101 - DNA, 17 bp, synthetic construct
  source 1..17: mol_type = other DNA
acgtggctggcattgca

SEQ ID NO: 102 - DNA, 23 bp, synthetic construct
  source 1..23: mol_type = other DNA
aagtggatcagtctctgcctcaa